(19) United States
(12) Patent Application Publication
(10) Pub. No.: US 2017/0348681 A1
(43) Pub. Date: Dec. 7, 2017

(54) ACTIVATION OF SUPPORTED OLEFIN METATHESIS CATALYSTS BY ORGANIC REDUCTANTS

(71) Applicants: ETH Zurich, Zurich (CH); Osaka University, Osaka (JP)

(72) Inventors: Christophe Coperet, Zurich (CH); Victor Mougel, Zurich (CH); Kazushi Mashima, Osaka (JP); Hayato Tsurugi, Osaka (JP); Haruki Nagae, Osaka (JP)

(21) Appl. No.: 15/536,446

(22) PCT Filed: Dec. 15, 2015
(86) PCT No.: PCT/CH2015/000185

§ 371 Date: Jun. 15, 2017

(30) Foreign Application Priority Data
Dec. 17, 2014 (EP) 14 004 251.6
Aug. 31, 2015 (EP) 15 002 559.1

(51) Int. Cl. B01J 31/16; C07C 67/465; C07C 6/06; C07C 6/04; B01J 37/16

(52) U.S. Cl. CPC B01J 31/1608; C07C 6/04; B01J 37/16; C07C 6/06; C07C 67/465; B01J 2231/543; B01J 2531/64; B01J 2531/66; C07C 2521/08; C07C 2531/22

(57) ABSTRACT

An organic reductant, in particular an organo silicon reductant suitable for activating supported catalysts of the type MOₙEₘ, wherein E is S and/or Se, in particular MOₙ, wherein M is W, Mo or Re, is described as well as its use in metathesis reactions. The reduced catalysts are able to metathesize olefins at low temperatures and are therefore also suitable for metathesis of functionalized olefins.

### CROSS REFERENCES TO RELATED APPLICATIONS

This application claims the priority of European patent applications nos. 14 004 251.6, filed Dec. 17, 2014 and 15 002559.1, filed Aug. 31, 2015 the disclosures of which are incorporated herein by reference in their entirety.

### TECHNICAL FIELD

The present invention concerns catalytic metathesis of alkenes, in particular low temperature activation—of preferably supported—Mo, W and Re oxide catalysts by organic reductants for low temperature metathesis of alkenes.

### BACKGROUND ART

One of the main drawbacks of metal oxide based alkene metathesis catalysts, especially tungsten oxide catalysis, is the need to be activated and to catalyze olefin metathesis at high temperatures only (typically at 200-400° C.). Consequently such catalysts are limited to high temperature operation and unfunctionalized olefins. In addition to be cost, energy and environmentally inefficient processes, the high temperature can induce non-desired reactions, such as isomerisation, and reduce the substrate scope.

Typical industrial olefin metathesis catalysts are based on the oxides of molybdenum, tungsten or rhenium supported on an inorganic refractory oxide such as silica, alumina, ceria, titania, zirconia or thoria or mixed oxides such as Al₂O₃—SiO₂. These catalysts are today prepared by several methods, which include the impregnation of a support with a precursor of the active species in solution, the co-precipitation of the metal precursor and the support, the mixing of the active metal material and the support material by mechanical means or the vapor deposition of the metal precursors.

An essential step in the activation of these catalysts consists in heating the catalysts at an elevated temperature in presence of air, an inert gas or the reactants.

To overcome the low activity of these systems, activation procedures have been developed, including alkylating agents, such as tetraalkyltin, trialkylaluminum or strained cyclic alkanes and alkenes, especially in the presence of nitrogeneous modifying reagents, high temperature treatments under alkene or inert gas flow and photoreduction processes.

In a more general perspective, activation of catalysts by reduction using organic reagents was proposed, such reductions typically taking place at elevated temperature (200-800° C.).

Some amount of reduction of the metal centers have been shown to be beneficial to the catalytic activity, and catalytic activity was found to be increased by the treatment of catalyst with reducing agents such as hydrogen, carbon monoxide and elemental metals.

In U.S. Pat. No. 5, 210,365 a disproportionation catalyst is disclosed that is obtained by forming a calcined composite comprising molybdenum or rhenium supported on an inorganic oxide support and contacting the calcined composite with an organosilane compound containing at least one silicon-hydrogen bond and/or at least one silicon-silicon bond per molecule like alkyl silanes, aryl silanes or respective disilanes. Such catalyst is described in the disproportionation of olefinic hydrocarbons.

Alternatively directly grafting a well-defined alkylidene complex or precursors of alkylidene on a support can generate active metathesis catalyst without activation procedure.

Attempts have also already en made using homogeneous catalysts instead of heterogeneous catalysts. Such catalysts are e.g. described in the thesis Schattenmann W. C. [8] and in JP 2013-14562 A.

Self-metathesis of allylsilanes in the presence of homogeneous ruthenium catalysts is described in Marciniec et al. [9].

In Saito [4] some silyl cyclodiene compounds are disclosed as reductants for transition metals in molecular complexes.

### Description

The problem to be solved by the present invention is therefore to provide a metathesis catalyst with higher activity and better performance, as well as good recoverability and regenerability.

This problem is solved by the improved heterogeneous alkene metathesis catalysts. Methods for their production are also described. Such catalysts can be obtained by reacting a supported metal oxide based alkene metathesis catalyst, such as tungsten oxide, rhenium oxide and/or molybdenum oxide, with an organic reductant either comprising at least one double bond in such proximity to one or more further double bonds that the oxidized compound is an aromatic system, like hexadiene resulting in benzene, or comprising at least one silyl group of the type SiX₂Y, in particular an organic reductant either comprising at least one double bond or at least one silyl group of the type SiX₂Y in such proximity to one or more further double bonds that the oxidized compound is an aromatic system, wherein in each silyl group of the type SiX₂Y,

each X is independently selected from H, R′, halogen, OR, NR₂, wherein
each R′ is independently selected from
   unsubstituted or substituted, linear or branched or cyclic C1 to C18 alkyl,
   unsubstituted or substituted linear or branched or cyclic C1 to C18 alkenyl,
   unsubstituted or substituted linear or branched or cyclic C1 to C18 alkynyl, or
   an unsubstituted or substituted aromatic group
each R is independently selected from H, R′, silyl of type —SiX₂Y

the Y of each silyl group can be the same or different and is selected from the group as defined for X or two Y together are —O—, or a single bond.

In some embodiments the Y of each silyl group can be the same or different and is selected from H, R′, halogen, OR and NR₂, wherein each R′ is as defined above and R is independently selected from H and R′, or two Y together are —O—, or a single bond.

Suitable catalysts are of the MOₙEₘ type with E being sulfur and/or selenium. A catalyst of MOₙEₘ type or an MOₙEₘ catalyst or a MOₙEₘ based catalyst are used synonymously and designate a catalyst with a metal center that prior to reduction is in direct contact with oxygen atoms/ions and possibly sulfur and/or selenium atoms/ions, such as ═O, —O⁻, —O-support, —OR, ═S, —S⁻, —S-support, —SR, ═Se, —Se⁻, —Se-support, —SeR. A preferred catalyst of the MOₙEₘ type is one with m=0, i.e. a catalyst of MOₙ type/a MOₙ catalyst/a MOₙ based catalyst. Also preferred are catalysts wherein the metal center is in contact with ═O, —O⁻, —O-support, ═S, —S⁻, —S-support, ═Se, —Se⁻, —Se-support, in particular ═O, —O⁻, —O-support.

S and Se comprising catalysts preferably are obtained starting from S and/or Se comprising precursors such as MS₂X₂ where M=W, Mo and Re and X=Cl and Br.

In proximity as used herein encompasses allylic and vinylic position, but also homoallylic or propargylic positions and preferably is allylic or vinylic position as shown by formula (I) below.

In order to efficiently act as reductants, the reductants of the present invention have to come in close contact with the solid catalyst and therefore are volatile or liquid under reaction conditions or soluble in a suitable solvent.

Such organic reductants can also be mixtures of organic reductants as defined herein. Preferred reductants comprise at least one double bond in proximity to at least one silyl group, more preferred an organic reductant of formula (I)

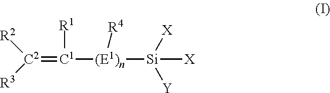

wherein

E¹ is selected from C—R⁵, N, P, As, or B

n is 0 or 1

R¹ to R⁴ and R⁵ are the same or different and are selected from the group comprising —H, —R′, silyl of type —SiX₂Y, —OR, —NR₂, halogens, —NO₂, phosphates, carbonates and sulfates, wherein in all the groups
each R′ is independently selected from the group comprising
   unsubstituted or substituted, linear or branched or cyclic C1 to C18 alkyl,
   unsubstituted or substituted linear or branched or cyclic C1 to C18 alkenyl,
   unsubstituted or substituted linear or branched or cyclic C1 to C18 alkynyl, or
   an unsubstituted or substituted aromatic group, in particular optionally aryl substituted C1 to C6 alkyl, such as methyl or butyl or benzyl or methylbenzyl, optionally alkyl like methyl substituted cyclohexyl, optionally alkyl like methyl substituted phenyl, e.g. tolyl,
each R is independently selected from the group comprising H, R′, silyl of type SiX₂Y,
   Or

R¹ and R² together form a —(E²)ₗ— chain that together with the C¹ and C² to which they are bound form a 4- to 12-membered ring, wherein
l is 2 to 10
   and/or

R³ and R⁴ together form a —(E²)ₘ— chain that together with the C² and E¹ to which they are bound form a 4- to 12-membered ring, wherein
m is 1 to 9 and wherein
each E² is independently from each other selected from the group comprising E¹R⁶, or O, or two adjacent E² are —CR⁷═CR⁸—, preferably in vinylic or allylic position with regard to one or more SiX₂Y group(s), wherein
E¹ is as defined above
R⁶, R⁷ and R⁸ are as defined for R⁵ or SiX₂Y

each X is independently selected from the group comprising H, R′, halogen, OR, NR₂, wherein
R′ and R are as defined above

each Y can be the same or different and is selected from the group as defined for X or two Y together are —O— or a single bond, wherein said —X₂Si—O—SiX₂-groups can be on adjacent E¹ and E² and/or on two adjacent E² and/or on adjacent E¹ and C1 and/or on adjacent E² and C², and/or on C¹and C², and/or on E¹ and E² spaced further apart and/or on E¹ and C² and/or on E² and C¹ spaced further apart and/or on E² and C² spaced further apart and/or on two E2 spaced further apart.

In preferred embodiments, at least one of the variables in formula (I) and much preferred all variables are selected from the following groups:

E¹ is selected from C—R⁵ and N

n is 1

R¹ to R⁴ and R⁵ are the same or different and are selected from the group comprising —H, —R′, silyl of type —SiX₃, wherein in all the groups
each R′ is independently selected from the group comprising
   unsubstituted or substituted, linear or branched or cyclic C1 to C6 alkyl,
   unsubstituted or substituted linear or branched or cyclic C1 to C6 alkenyl,
   unsubstituted or substituted linear or branched or cyclic C1 to C6 alkynyl or
   an unsubstituted or substituted up to 6 membered aromatic group,
each R is independently selected from the group comprising H, R′, silyl of type —SiX₃,
   or

R¹ and R² together form a —(E²)₁— chain that together with the C¹ and C² to which they are bound form a 6-membered ring, wherein
l is 4
   and/or

R³ and R⁴ together form a —(E²)ₘ— chain that together with the C² and E¹ to which they are bound form a 5 to 8-membered ring, wherein
m is 2 to 5 and wherein
each E² is independently selected from the group comprising E¹R⁶, or two adjacent E² are —CR⁷═CR⁸—, preferably in vinylic or allylic position with regard to one or more SiX₃ group(s), wherein
E¹ is as defined above
R⁶, R⁷ and R⁸ are as defined for R⁵ or SiX₃

each X is independently selected from the group comprising H and R′, wherein
R′ is as defined above.

In even more preferred embodiments each

R′ is independently an optionally aryl substituted C1 to C6 alkyl group such as a methyl group or a butyl group or a benzyl group or a methylbenzyl group, an optionally alkyl substituted cyclohexyl group like a methyl substituted cyclohexyl group, an optionally alkyl substituted phenyl group like a methyl substituted phenyl group, e.g. a tolyl group,

and/or

E² is E¹R⁶ wherein R⁶ is —SiX₂Y wherein X and Y are as defined above and preferably are hydrogen or methyl or —O—.

In much preferred embodiments, the compounds of formula (I) are silyl groups substituted homo or hetero cycles comprising at least one silyl group in proximity (preferably allylic or vinylic position, most preferred allylic position) to a double bond such that upon reduction one or more aromatic rings are formed.

Specific groups falling under formula (I) are e.g. cyclohexadiene moieties substituted with one or more, preferably two trialkylsilyl groups or 1,4-diazacyclohexadiene moieties substituted with one or more, preferably two silyl groups, in particular groups of formula (II)

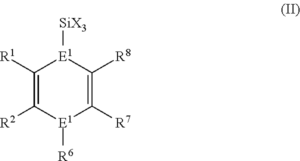

wherein R¹, R², R⁶, R⁷ and R⁸ are as defined above and presently preferred R¹, R², R⁷ and R⁸ are hydrogen or methyl and preferred R⁶ is SiMe₃.

Further specific groups falling under formula (I) are e.g. compounds of one of formulas (III) to (VII).

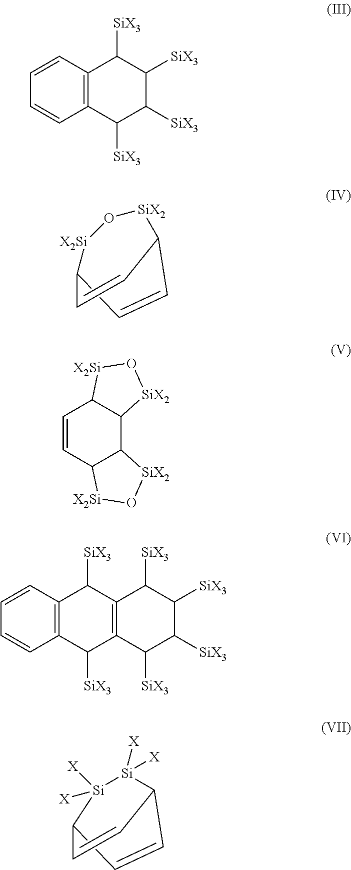

For simplicity reasons formulas (III) to (V) have been drawn without indicating the possibility that in particular the SiX3 carrying position might be N instead of C and that the C's might be substituted. These possibilities, however, are also encompassed by the present invention although the compounds of the formulas as indicated are the presently preferred ones.

Compounds of formula (II) encompass the following compounds later on referred to as Red1, Red2, Red3 and Red4.

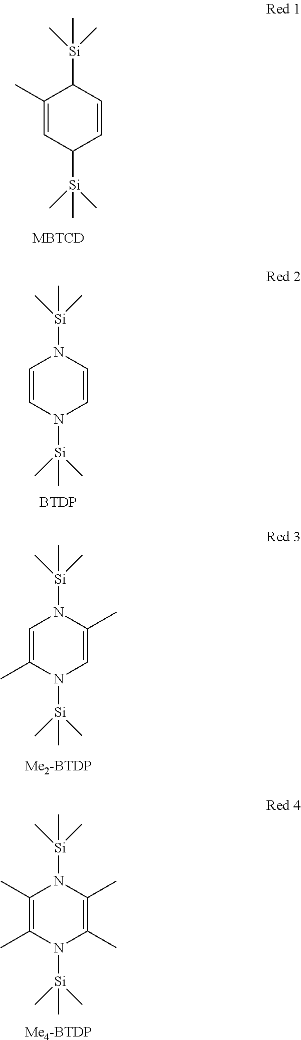

The alkyl groups in the trialkylsilyl groups are not critical but preferably are independently linear or branched or cyclic or aromatic C1 to C6 groups, more preferred all alkyl or cycloalkyl or aromatic groups are the same, such as methyl groups.

The reductant can be added to the catalysts before the methathesis reaction is performed or more conveniently directly in the presence of the alkene substrate. These catalysts present significantly higher conversion rates and selectivities than the parent materials before reduction. The much greater activity of the reduced catalysts allows running reaction at significantly lower temperature, reducing or even eliminating non desired side-reactions and allowing the use of functionalized alkenes such as alkenes substituted with a group selected from ethers, esters, amines, amides, imides, alcohols, ketones, aldehydes, thiols, acetals, thioacetals, boronic acids, boronic esters, silyl ethers, alkyl silyls, halogeno alkyls, alkyl phosphine, aluminum alkyl, carboxylates, nitro, phosphates and sulfonates.

The catalysts of this invention consist of a metal oxide component, such as tungsten oxide and/or molybdenum oxide and/or rhenium oxide, supported on a heterogeneous support, which is treated by an organic reductant that is an organic compound comprising at least one double bond and/or at least one silyl group as defined above and preferably is an organosilicon reductant of formula (I). Suitable heterogeneous supports comprise silica, alumina, ceria, titania, niobia, thoria, zirconia or mixed oxides such as Al₂O₃—SiO₂.

The molar ratio of reductant to metal will typically range from 0.0001:1 to 10000:1, preferably 0.01:1 to 10:1, more preferred 0.1:1 to 5:1. These ranges take into account that in many catalysts, in particular many of the commercially available catalysts, not catalytically active metal centers, notably burried inside crystalites of the metal oxide and not accessible to the reductant or the substrate are present, in some catalyst in a large excess with regard to the active metal centers. With regard to possibly catalytically active centers a ratio of reductant to metal of about 0.5:1 to 2:1 is preferred.

The reductant can be added to the catalyst in pure form or in solution in organic solvent to generate an active catalyst, or the reductant can be added together with or after the olefin substrate to generate the active catalyst in situ.

To conduct metathesis reactions employing the catalysts of this invention, a wide range of reaction conditions can be used. In general, the reaction conditions are similar to those described in the prior art, and can consist in batch conditions or flow conditions.

The reduction as well as the metathesis reaction can be carried out in the presence or in the absence of an inert solvent, in liquid phase or in gas phase. Reaction temperatures can vary between −20° C. and 500° C., the reaction being generally optimal in the 40-250° C. range such as at about 70° C. The organic solvent—if used—can be any aprotic organic solvent or mixture of such solvents, although for the reduction reaction polar solvents have been found beneficial. The solvent is e.g. chosen in dependency of the reaction temperature, e.g. benzene or chloroalkanes for reactions performed below 80° C., toluene or trifluorotoluene for reactions up to 110° C. and chlorobenzenes for higher reaction temperatures.

The reduction as well as the metathesis reaction are generally conducted under inert atmosphere, with precautions to exclude exposure to moisture and oxygen. The sensitivity to oxygen and moisture of the catalysts of the present invention in the presence of reductant seems less critical than for known catalysts, nevertheless the reactions should be performed in oxygen-free and water-free environment, which means less than about 50 ppm of remaining oxygen and water. Within these conditions, quantitative conversions and selectivity were observed even at low level of metal to olefin loading, typically chosen in the range 0.00001-1 mole of metal per mole of substrate, usually in the range 0.00001-0.1 mole of metal per mole of substrate.

Synthesis of catalysts and investigation of the catalytic properties are described in the examples presented further below.

The data given below, in particular in the experimental part, clearly demonstrate the significant advantage obtained with the catalysts treated with the reductants of the present invention, in particular the organosilicon reductants of formula (I). As an example, an unactivated tungsten oxide catalyst did not show any activity in the conditions tested, while catalysts treated with the organic reductants, in particular the organosilicon reductants of formula (I) demonstrated high activity in alkene metathesis. Highest activity was obtained when the organosilicon reagent was added together with the olefin substrate but indepedant reduction was also shown to result in increased activity.

The reduction step in the inventive process appears to be essential. As organic reductant any compound with at least two double bonds as defined above or a combination of at least one double bond and at least one silyl group seems suitable, however a combined organosilicon reagent of formula (I) is preferred. In a more preferred embodiment the reductants comprise a cyclohexadiene moiety or a diaza cyclohexadiene moiety. In view of the results obtained, reductants that are able to form aromatic systems are especially suited.

Different catalyst materials can be activated using the reductant of the present invention, in particular industrially relevant catalysts such as WO₃/SiO₂ and MoO₃/SiO₂ and ReₓO_{y}/SiO₂and ReₓO_{y}/Al₂O₃ or such catalysts on other supports selected from e.g. SiO₂ or Al₂O₃ or Al₂O₃—SiO₂ or other metal oxides from the group mentioned above, like ceria, titania, zirconia and niobia.

The exact structure of the catalysts of the present invention is not yet fully known, however, if silyl groups comprising reductants are used, silyloxy groups (—O—SiX₂Y) can be found attached to the supported activated, i.e. at least partially reduced, MOₙ catalyst. Said supported catalyst—according to present information—has the following general formula (VIII),

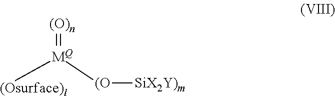

wherein

Q is the valence of the metal which may be a mixed valence due to differently reduced metal centers

l is 1 to 4,

n is 0 to 2,

l+m+2n=Q and

each X is independently selected from H, R′, halogen, OR, NR₂, wherein
each R′ is independently selected from
   unsubstituted or substituted, linear or branched or cyclic C1 to C18 alkyl,
   unsubstituted or substituted linear or branched or cyclic C1 to C18 alkenyl,
   unsubstituted or substituted linear or branched or cyclic C1 to C18 alkynyl or
   an unsubstituted or substituted aromatic group, in particular optionally aryl substituted C1 to C6 alkyl such as methyl or butyl or benzyl or methylbenzyl, optionally alkyl like methyl substituted cyclohexyl, optionally alkyl like methyl substituted phenyl, such as tolyl, and
each R is independently selected from the group consisting of H, R′ and silyl of the type —SiX₂Y, wherein
   R′ is as defined above and
   the Y of each silyl group can be the same or different and is selected from the group as defined for X or two Y together are —O— or a single bond.

Usually the compound of formula (VIII) is generated using the reductant as described here and thus X and Y in general are as found in the reductant.

In some specific embodiments the Y of each silyl group can be the same or different and is selected from H, R′, halogen, OR and NR₂, wherein each R′ is as defined above and R is independently selected from H and R′, or two Y together are —O—, or a single bond.

The reductants and methods of the present invention allow a very efficient reduction that works in solution phase and results in the activation of poorly active alkene metathesis catalysts in one step at low temperature. The catalysts thus activated present activities several orders of magnitudes greater than the parent/precursor materials. Moreover, the use of organic reductants, in particular organosilicon reductants of formula (I), allows to limit the presence of byproducts on the surface, generally obtained when alkali metals are used as reductant, and thus the generation of active sites for the competitive isomerisation of the olefin substrate is reduced. Moreover, the inventive catalysts present a significant advantage over the reduction with gases such as olefin or hydrogen at high temperatures (above 300° C.), due to the lower temperature of activation required according to the present invention and since the use of dihydrogen favors undesired reactions such as hydrogenation of the alkene substrate. It also makes the inventive approach compatible with functionalized olefins.

Another advantage of the catalysts of the present invention is that they can readily be recycled. If they lose activity they can be reactivated by again treating them with one of the reductants of the present invention, either in a separate regeneration reaction or in situ.

Other advantageous embodiments are listed in the dependent claims as well as in the description below,

### BRIEF DESCRIPTION OF THE FIGURES

The invention will be better understood and objects other than those set forth above will become apparent from the following detailed description thereof. Such description makes reference to the annexed drawings.

FIG. 1. Thermal ellipsoid plot at the 50% probability of [WO₂(OSi(OtBu)₃)₂(DME)]. Hydrogen atoms have been omitted and only one of the three independent molecules in the asymmetric unit has been represented for clarity.

FIG. 2. FTIR transmission spectra of [(≡SiO)WO₂(OSi(OtBu)₃)]

FIG. 3. EXAFS spectrum of WO₂(OSi(OtBu)₃)₂(DME).

FIG. 4. ¹H NMR spectrum (400 MHz, spinning rate 10 kHz, 4 mm rotor) of [(≡SiO)WO₂(OSi(OtBu)₃)] (*: spinning side bands).

FIG. 5. ¹³C CP-MAS NMR spectrum (400 MHz, spinning rate 10 kHz, 4 mm rotor) of [(≡SiO)WO₂(OSi(OtBu)₃)] (d1=2s, contact time=2 ms).

FIG. 6. EXAFS spectrum of WO₂(OSi(OtBu)₃)₂(DME) grafted onto [SiO₂₋₇₀₀], i.e. [(≡SiO)WO₂(OSi(OtBu)₃)].

FIG. 7. FTIR transmission spectra of [(≡SiO)₂WO₂] (black line, (a)) compared with the parent [(≡SiO)WO₂(OSi(OtBu)₃)] complex (grey line, (b)).

FIG. 8. EXAFS spectrum of WO₂(OSi(OtBu)₃)₂(DME) grafted and thermally decomposed onto [SiO₂₋₇₀₀], i.e. [(≡SiO)₂WO₂].

FIG. 9. FTIR of the materials [(≡SiO)₂WO₂](Red1)_{0.5}, (a), [(≡SiO)₂WO₂](Red2)_{0.5}, (b), [(≡SiO)₂WO₂](Red3)_{0.5}, (c) and [(≡SiO)₂WO₂](Red4)_{0.5}, (d).

FIG. 10. FTIR of the materials [(≡SiO)₂WO₂](Red4)_{0.5}, (d), [(≡SiO)₂WO₂](Red4)₁, (c), [(≡SiO)₂WO₂](Red4)₂, (b), and [(≡SiO)₂WO₂](Red4)₃, (a).

FIG. 11. FTIR of the materials WO₂Cl₂(DME)/SiO₂, (a), [(≡SiO)₂WO₂]_{Cl}, (b) and [(≡SiO)₂WO₂]_{Cl}(Red4)₂, (c).

FIG. 12. EXAFS spectrum of WO₂Cl₂(DME)/SiO₂ thermally decomposed under vacuum, i.e. [(≡SiO)₂WO₂]_{Cl}.

FIG. 13: FTIR of the materials [(≡SiO)MoO₂{OSi(O^{t}Bu)₃}] (a) and [(≡SiO)MoO₂] (b).

FIG. 14. EXAFS spectrum of MoO₂[OSi(O^{t}Bu)₃]₂ (a), [(≡SiO)MoO₂{OSi(O^{t}Bu)₃}] (b) and [(≡SiO)MoO₂] (c).

FIG. 15. Conversion vs time, cis-4-nonene homometathesis, 0.1 mol % W, 70° C. for [(≡SiO)2WO₂](Red4)₂ (diamonds), [(≡SiO)₂WO₂](Red4); (empty circles), [(≡SiO)2WO₂](Red4)_{0.5} (crosses), [(≡SiO)₂WO₂](Red1)₁ (empty squares) and [(≡Si(₂WO₂]+0.2 mol % Red4 (triangles).

FIG. 16. Conversion vs time, cis-4-nonene homometathesis, 0.1 mol % W, 70° C. for [(≡SiO)₂WO₂] in presence of two equivalents of the following reagents: Red4 (diamonds), allyltrimethyilane (squares), cyclohexadiene (triangles), vinyltriethoxysilane (crosses) and 1,4-bistrimethylsilylbenzene (stars).

FIG. 17. Conversion, diethyl diallylmalonate ring closing metathesis, 0.1 mol % W, 70° C. for [(≡SiO)₂WO₂]: 90h after initial addition of 2 equiv. of Red4 (a) and 90h after second addition of 2 equiv. of Red4 (b).

FIG. 18. Conversion vs time, cis-4-nonene homometathesis, 0.1 mol % W, 70° C. for [(≡SiO)₂WO₂]_{Cl} in presence of two equivalents of Red4 (diamonds) and [(≡SiO)₂WO₂]_{Cl}(Red4)₂ (squares).

FIG. 19. Conversion vs time, cis-4-nonene homometathesis, 0.1 mol % W, 30° C. for [(≡SiO)MoO₂] in presence of two equivalents of Red4 (squares).

FIG. 20. Conversion vs time, cis-4-nonene homometathesis, 0.1 mol % W, 70° C. for Re₂O₇/SiO₂ in absence (diamonds) and in presence of two equivalents of Red1 (squares).

### MODES FOR CARRYING OUT THE INVENTION

### Preliminary Remarks on Nomenclature

MOₙ/support designates any of the supported tungsten oxide, molybdenum oxide or rhenium oxide catalysts on any metal oxide support as defined above.

The designation catalyst/support indicates that the structure of the supported catalyst is not fully determined or that differently bound catalytical sites can be present.

(≡SiO) means an isolated siloxy group of the silica surface or three bonds ≡ of surface silica to the bulk, respectively.

[(≡SiO)ₘMOₙ] means a determined structure with m siloxy groups bound to one metal center M.

### Experimental Part

### A) General Procedures

All experiments were carried out under dry and oxygen free argon atmosphere using either standard Schlenk or glove-box techniques. Pentane, toluene and diethyl ether were purified using double MBraun SPS alumina column, and were degassed using three freeze-pump-thaw cycles before being used. Dimethoxyethane (DME) and tetrahydrofuran (THF) were distilled from Na/Benzophenone. Silica (Aerosil Degussa, 200 m²g⁻¹) was compacted with distilled water, calcined at 500° C. under air for 4 h and treated under vacuum (10⁻⁵ mbar) at 500° C. for 6 h and then at 700° C. for 10 h (support referred to as SiO₂₋₍₇₀₀₎) and contained 0.26 mmol of OH per g as measured by titration with PhCH₂MgCl. All infrared (IR) spectra were recorded using a Bruker spectrometer placed in the glovebox, equipped with OPUS software. A typical experiment consisted in the measurement of transmission in 32 scans in the region from 4000 to 400 cm⁻¹. The ¹H and ¹³C-NMR spectra were obtained on Bruker DRX 200, DRX 250 or DRX 500 spectrometers. The solution spectra were recorded in C₆D₆ at room temperature. The ¹H and ¹³C chemical shifts are referenced relative to the residual solvent peak. Compounds WO₂Cl₂(DME),[1] WOCl₄,[2] [MoO₂(OSi(OtBu)₃)₂],[6] 1-methyl-3,6-bis(trimethylsilyl)-1,4-cyclohexadiene (Red1),[3] 1,4-bis(trimethylsilyl)-1,4-diaza-2,5-cyclohexadiene (Red2), 2,5-dimethyl-1,4-bis(trimethylsilyl)-1,4-diaza-2,5-cyclohexadiene (Red3), 2,3,5,6-tetramethyl-1,4-bis(trimethylsilyl)-1,4-diaza-2,5-cyclohexadiene (Red4),[4] were synthesized according to literature procedures. LiOSi(OtBu)₃ was obtained by deprotonation of HOSi(OtBu)₃ with n-BuLi according the published procedure.[6] Ammonium metatungstate and ammonium heptamolybdate hydrates were purchased from Fiuka and used without purification. WO₃/SiO₂ and MoO₃/SiO₂ were synthesized by incipient wetness impregnation followed by calcination at 450° C.[5] It was determined by elemental analysis to contain 7.12% W in mass for WO₃/SiO₂ and 7% Mo in mass for MoO₃/SiO₂. Re₂O₇/SiO₂ was prepared according to a method described in [7]. Unless otherwise stated, reductions and catalytic tests were carried out at 70° C.

### B) Syntheses and Characterisation of the Materials

### B) I) Synthesis of the Molecular Precursors Involving Alkoholate Comprising Precursors:

Synthesis of [WO₂(OSi(OtBu)₃)₂(DME)]

[WO₂(OSi(OtBu)₃)₂(DME)] was synthesized using a modification of the procedure described by Tilley.[6]

A solution of LiOSi(OtBu)₃ (2.87 g, 10.6 mmol, 2 eq.) in cold toluene (15 mL, −40° C.) was added dropwise to a suspension of WO₂Cl₂(DME) (2 g, 5.3 mmol, 1. eq.) in toluene (20 mL, −78° C.) containing 200 μL of DME under vigorous stirring. After 1 hour stirring at −78° C. and 2 hours at room temperature, the solution was filtered through a short Celite® pad to afford a colorless solution. Crystallization of the product from this solution at −40° C. afforded 3.2 g (3.8 mmol, 72%) of the title product as large colorless needle shaped crystals suitable for XRD (collected in two crops).

¹H-NMR (300 MHz, C6D6) δ1.38 (s, 54, (OtBu)₃), 3.15 (s, 6, DME), 3.33 (s, 4, DME).

IR (KBr, cm⁻¹): 703(m), 830(m), 858(m), 902(m), 948(m), 962(m), 1028(m), 1066(s), 1191(m), 1243(m), 1366(m), 1390(m), 1473(w), 2975(m).

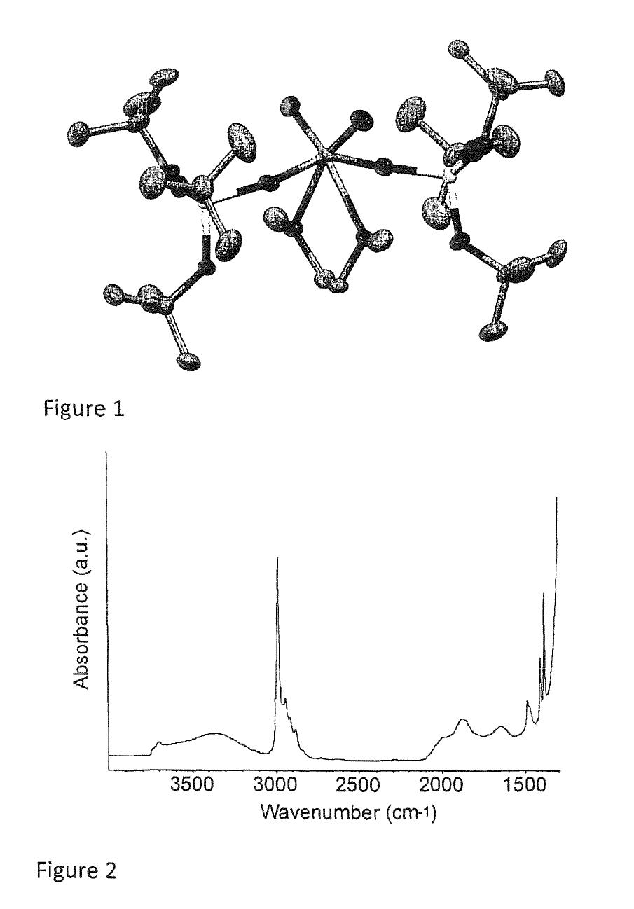

The XRD structure is shown in FIG. 1, Selected bonds for [WO₂(OSi(OtBu)₃)₂(DME)] are listed in Table 1 (distances are given in Å) and crystallographic data for [WO₂(OSi(OtBu)₃)₂(DME)] are presented in Table 2.

TABLE 1
(distances in Å):

| Structural parameters | [WO₂(OSi(OtBu)₃)₂(DME)] |
|---|---|
| W1 - O1 | 1.719 (5) |
| W1 - O2 | 1.716 (5) |
| W1 - O3 | 1.924 (4) |
| W1 - O4 | 1.928 (4) |
| W1 - O5 | 2.332 (4) |
| W1 - O6 | 2.344 (4) |

TABLE 2

| Formula | C₁₁₉H₂₆₄O₄₈Si₈W₄ | |
|---|---|---|
| Crystal size (mm) | 0.7 × 0.2 × 0.2 | |
| cryst syst | Tetragonal | |
| space group | I41 | |
| volume (Å³) | 16779.5 | (4) |
| a (Å) | 23.6586 | (3) |
| b (Å) | 23.6586 | (3) |
| c (Å) | 29.9778 | (5) |
| α (deg) | 90 | |
| β (deg) | 90 | |
| γ (deg) | 90 | |
| Z | 4 | |
| formula weight (g/mol) | 3423.44 | |
| density (g cm⁻³) | 1.355 | |
| F(000) | 7075.3 | |
| temp (K) | 150.0 | (3) |
| total no. reflections | 30830 | |
| unique reflections [R(int)] | 23689 | [0.1046] |

| | |
|---|---|
| Final R indices [I > 2σ(I)] | R₁ = 0.0641, wR₂ = 0.1208 |
| Largest diff. peak and hole (e.A⁻³) | 2.62/−3.91 |
| GOF | 1.050 |

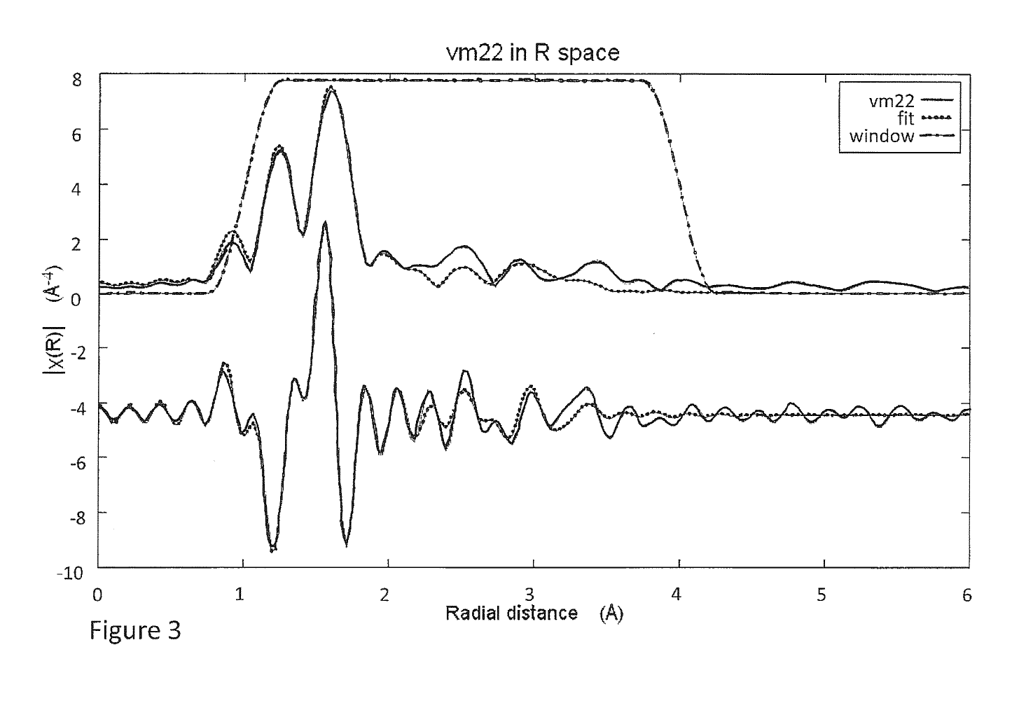

An EXAFS (extended X-ray absorption fine structure) spectrum of WO₂(OSi(OtBu)₃)₂(DME) is shown in FIG. 3 and the relevant data are listed below in Table 3.

TABLE 3

| Scatterer | N | S02 | r model | delr | R | ss\hat{ }2 | enot |
|---|---|---|---|---|---|---|---|
| Oa | 2 | 1 | 1.717 | 0.030 | 1.74 | 0.00493 | 4.52 |
| Ob | 2 | 1 | 1.926 | −0.016 | 1.91 | 0.00081 | 4.52 |
| Oc | 2 | 1 | 2.338 | 0.038 | 2.38 | 0.0224 | 4.52 |
| O 2x scatter | 2 | 1 | 3.092 | −0.025 | 3.07 | 0.00978 | 4.52 |
| O 2x scatter | 8 | 1 | 3.204 | −0.025 | 3.18 | 0.00978 | 4.52 |
| C | 2 | 1 | 3.209 | 0.156 | 3.36 | 0.00978 | 4.52 |
| C | 2 | 1 | 3.270 | 0.156 | 3.42 | 0.00978 | 4.52 |
| Si | 1 | 1 | 3.348 | 0.156 | 3.50 | 0.00978 | 4.52 |
| Si | 1 | 1 | 3.378 | 0.156 | 3.53 | 0.00978 | 4.52 |

Synthesis of [(≡SiO)WO₂(OSi(O^{t}Bu)₃)]

A solution of 1 g of WO₂[OSi(OtBu)₃]₂(DME) (1.25 mmol, 1.05 equiv.) in benzene (6 mL) was added to a suspension of SiO₂₋₍₇₀₀₎ (4.61 g, 1.19 mmol, 1 equiv.) in benzene (3 mL) at room temperature. The suspension was slowly stirred at room temperature for 12 h. The white solid was collected by filtration, and was washed by five suspension/filtration cycles in benzene (5×2 mL). The resulting solid was dried thoroughly under high vacuum (10⁻⁵ mbar) at room temperature for 3h to afford 4.55 g of the title compound. All the filtrate solutions were collected and analyzed by ¹H NMR spectroscopy in C₆D₆ using ferrocene as internal standard, indicating that 0.7 mmol of (^{t}BuO)₃SiOH and 0.47 mmol of DME were released upon grafting (0.60 equiv. (^{t}BuO)₃SiOH and 0.40 equiv.DME). Additional 0.65 mmol of DME were quantified in the volatiles collected upon high vacuum drying, indicating that >95% of DME was not retained on the silica surface.

Elemental Analysis: W 3.36%, C 2.77%, H 0.74% corresponding to 12.6 C/W (12 expected), 40.2 H/W (39 expected).

IR (KBr, cm⁻¹): 1369 (s), 1393 (m), 1474 (w), 2937 (m, sh), 2979 (s).

The FTIR transmission spectra of [(≡SiO)WO₂(OSi(OtBu)₃)] is shown in FIG. 2.

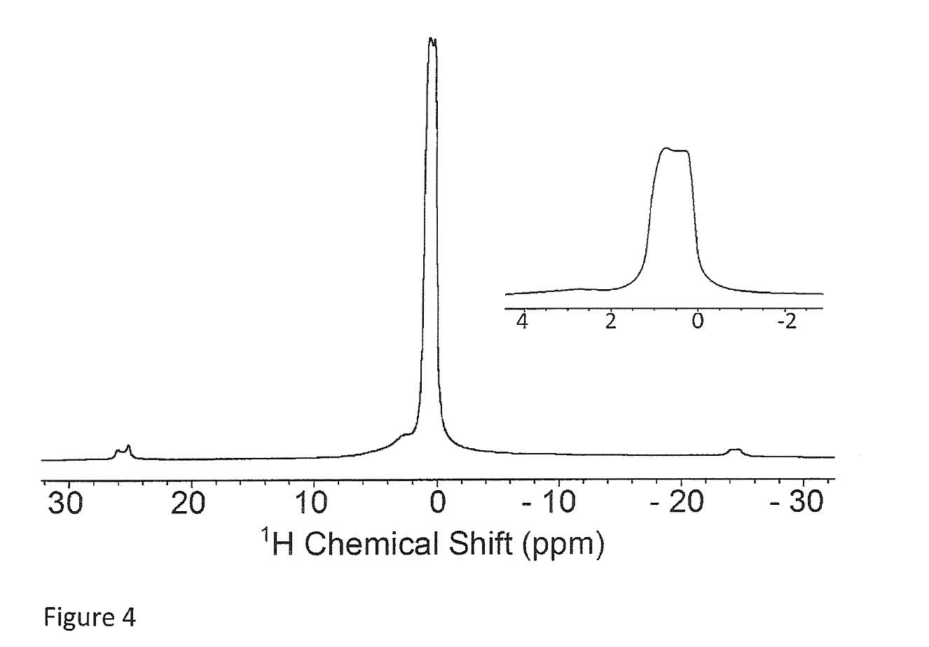

The ¹H NMR spectrum (400 MHz, spinning rate 10 kHz, 4 mm rotor) of [(≡SiO)WO₂(OSi(OtBu)₃)] (*: spinning side bands) is shown in FIG. 4.

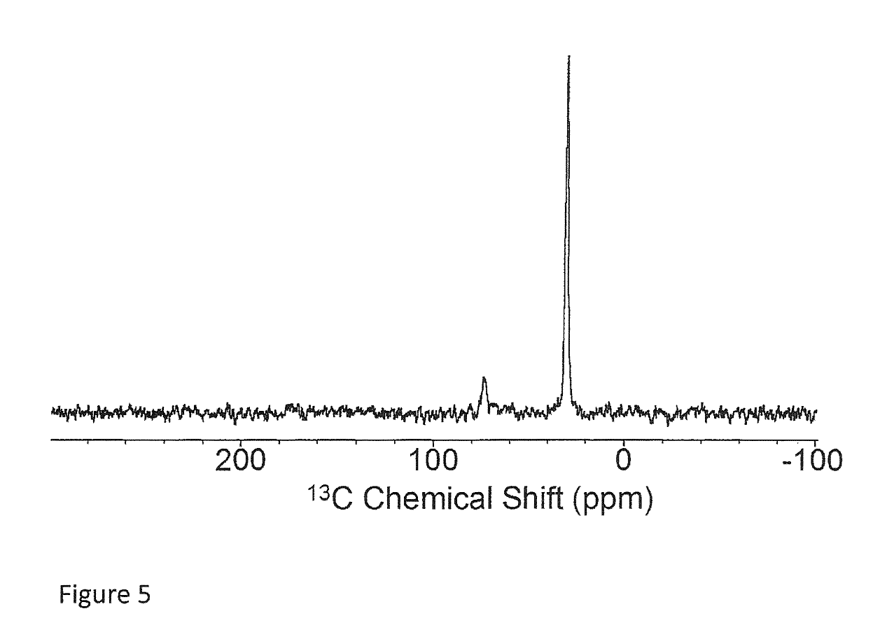

The ¹³C CP-MAS NMR spectrum (400 MHz, spinning rate 10 kHz, 4 mm rotor) of [(≡SiO)WO₂(OSi(OtBu)₃)] (d1=2s, contact time=2 ms) is shown in FIG. 5.

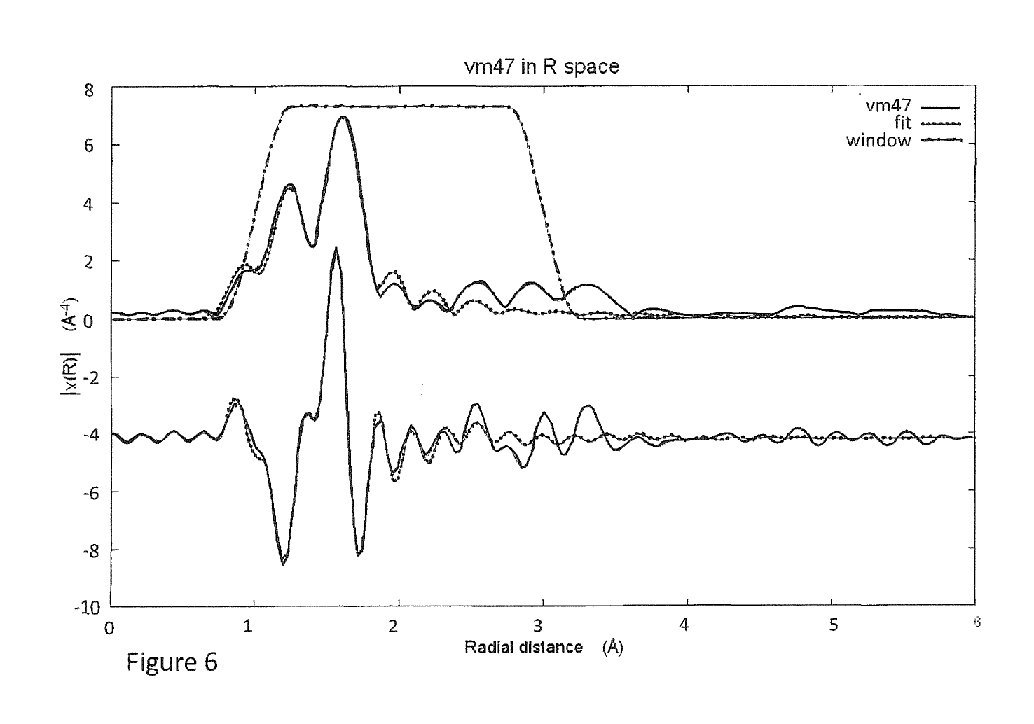

An EXAFS (extended X-ray absorption fine structure) spectrum of WO₂(OSi(OtBu)₃)₂(DME) grafted onto [SiO₂₋₇₀₀], [(≡SiO)WO₂(OSi(OtBu)₃)], is shown in FIG. 6 and the relevant data are listed below in Table 4.

TABLE 4

| Scatterer | N | S02 | r model | delr | R | ss\hat{ }2 | enot |
|---|---|---|---|---|---|---|---|
| Oa | 2 | 1 | 1.717 | 0.0407 | 1.76 | 0.00604 | 6.87 |
| Ob | 2 | 1 | 1.926 | −0.00354 | 1.92 | 0.00118 | 6.87 |
| O 2x scatter | 2 | 1 | 3.092 | −0.103 | 2.99 | 0.0162 | 6.87 |
| O 2x scatter | 8 | 1 | 3.204 | −0.103 | 3.10 | 0.0162 | 6.87 |

Thermal Decomposition of [(SiO)WO₂(OSi(OtBu)₃)]: Preparation of [(≡SiO)₂WO₂]

[(≡SiO)WO₂(OSi(OtBu)₃)] (3.0 g) was loaded into a reactor and placed under high vacuum (10⁻⁵ mbar) and heated to 200° C. (1° C./min) and kept at 200° C. for 3 h, then heated to 400° C. (1° C./min) and kept at 400° C. for 6 h. The reactor was cooled to ambient temperature under vacuum, and [(≡SiO)₂WO₂] was stored in an Ar filled glovebox. The volatiles liberated during this process were quantified by ¹H NMR in C6D6 with ferrocene as an internal standard as 2.5 equiv of isobutylene, 0.6 equiv. of water and 0.8 equiv of tBuOH per surface W complex.

Elemental analysis: W 3.56%.

IR (KBr, cm⁻¹): 3746 (s).

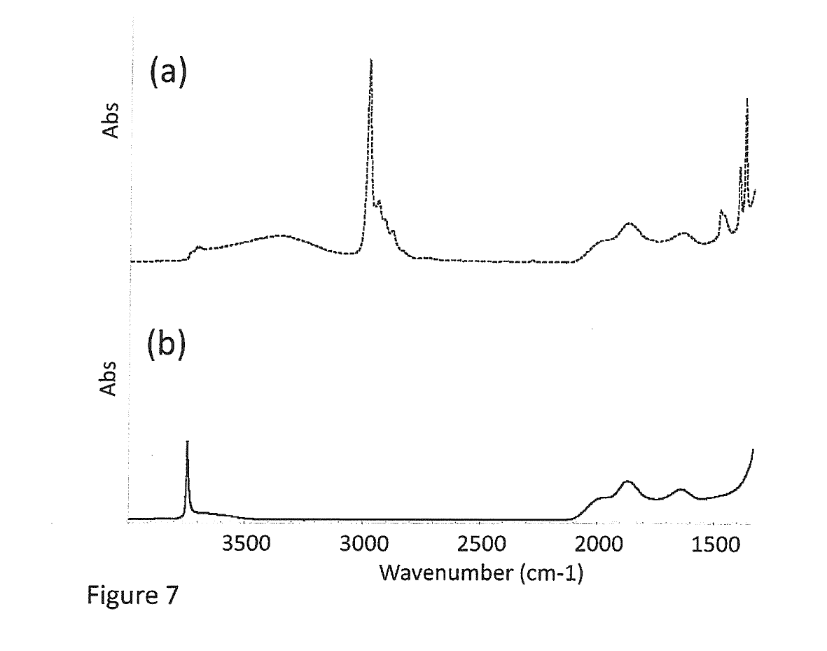

FTIR transmission spectra of [(≡SiO)₂WO₂] (grey line, (b)) compared with the parent [(≡SiO)WO₂(OSi(OtBu)₃)] complex (black line, (a)) is shown in FIG. 7.

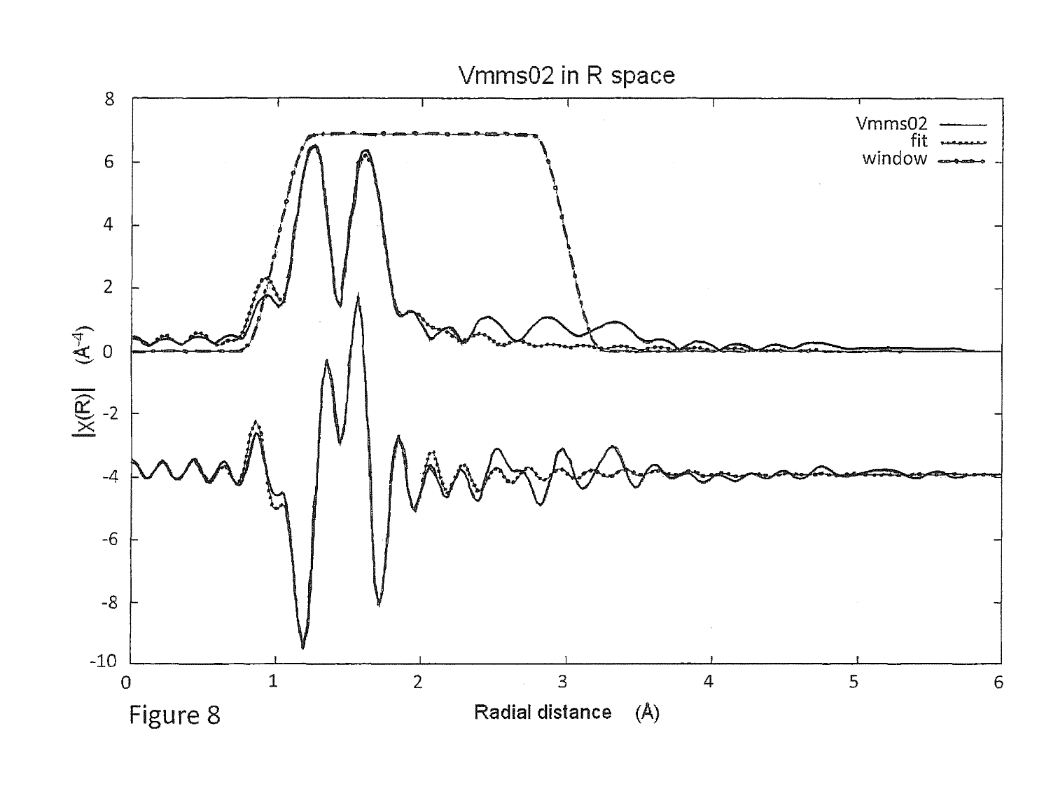

An EXAFS (extended X-ray absorption fine structure) spectrum of WO₂(OSi(OtBu)₃)₂(DME) grafted and thermally decomposed onto [SiO₂₋₇₀₀], [(□SiO)₂WO₂], is shown in FIG. 8 and the relevant data are listed below in Table 5.

TABLE 5

| Scatterer | N | S02 | r model | delr | R | ss\hat{ }2 | enot |
|---|---|---|---|---|---|---|---|
| O | 2 | 1 | 1.717 | 0.0083 | 1.73 | 0.00334 | 6.50 |
| O | 2 | 1 | 1.926 | −0.022 | 1.90 | 0.00122 | 6.50 |

For the reduction of the materials, organosilicon reductants of the following formula (II) were primarily used:

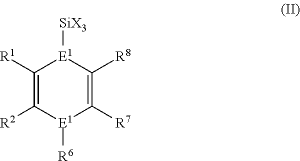

wherein E¹ is CH or N, R¹, R², R⁷ and R⁸ are H or CH₃ and R⁶ is SiX3 and X is methyl.

In particular the following reductants were used in the Examples:

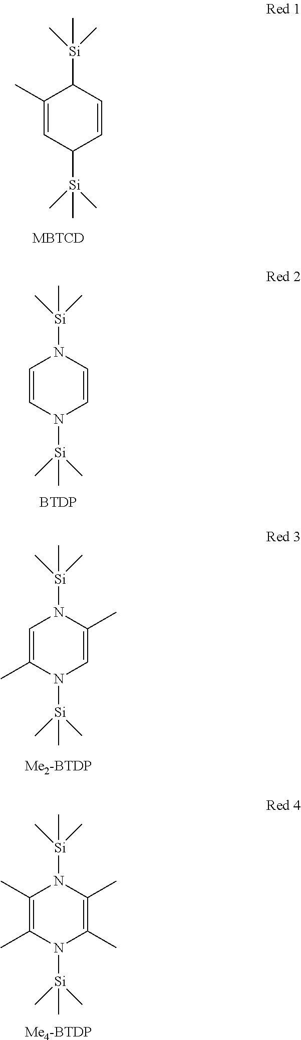

The general reaction scheme using such compounds of formula (II) is as follows:

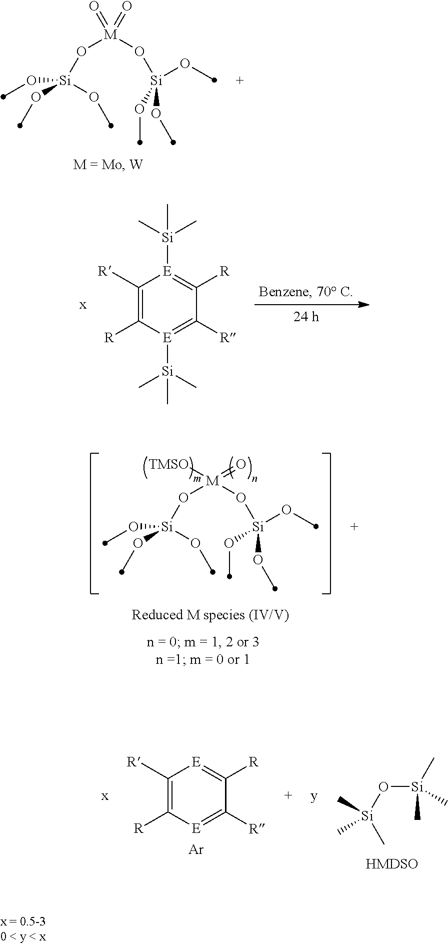

Representative Procedure: Reduction of [(≡SiO)₂WO₂] with 1 Equiv. of 2,6 trimethylsilyl tetramethyl diazacyclohexadiene (Red4).

A solution of 5.4 mg of Red4 (19 μmol, 1 equiv.) in benzene (0.5 mL) was added to a suspension of [(≡SiO)₂WO₂] (100 mg, 19 μmol) in benzene (0.5 mL) at room temperature. The suspension was slowly stirred at 70° C. for 12h, resulting in color change of the material from colorless to dark violet. The solid was collected by filtration, and was washed by four suspension/filtration cycles in benzene (4×1 mL). The resulting dark violet solid was dried thoroughly under high vacuum (10⁻⁵ mbar) at room temperature for 3h to afford 90 mg of the title compound. All the filtrate solutions were collected and analyzed by ¹H NMR spectroscopy in C₆D₆ using ferrocene as internal standard, indicating full consumption of Red4 and that 0.011 mmol of 1,2,4,5-tetramethylpyrazine and 0.006 mmol of hexamethyldisiloxane (HMDSO) were released upon reacting (0.55 equiv. 1,2,4,5-tetramethylpyrazine).

Reduction of [(≡SiO)₂WO₂] with 1 Equiv. of Reductant Red1-Red4:

The reductions were carried out following the procedure above. 100 mg of [(≡SiO)₂WO_{2]} were reduced with 1 equiv. of the four reductants represented above. Analyses of the filtrate by NMR are summarized in Table 6:

TABLE 6

| | | | | Colour | |
|---|---|---|---|---|---|
| | Consumption | Aromatized | | of the | |
| Reductant | of Red. | Bp (Ar) | HMDSO | material | Material name |
| Red1 | 10% | 10% | 1% | Blue | [(≡SiO)₂WO₂](Red1)₁ |
| Red2 | 100% | 1% | 2% | Dark | [(≡SiO)₂WO₂](Red2)₁ |
| | | | | violet | |
| Red3 | 100% | 33% | 4% | Dark | [(≡SiO)₂WO₂](Red3)₁ |
| | | | | violet | |
| Red4 | 100% | 55% | 3% | Dark | [(≡SiO)₂WO₂](Red4)₁ |
| | | | | violet | |

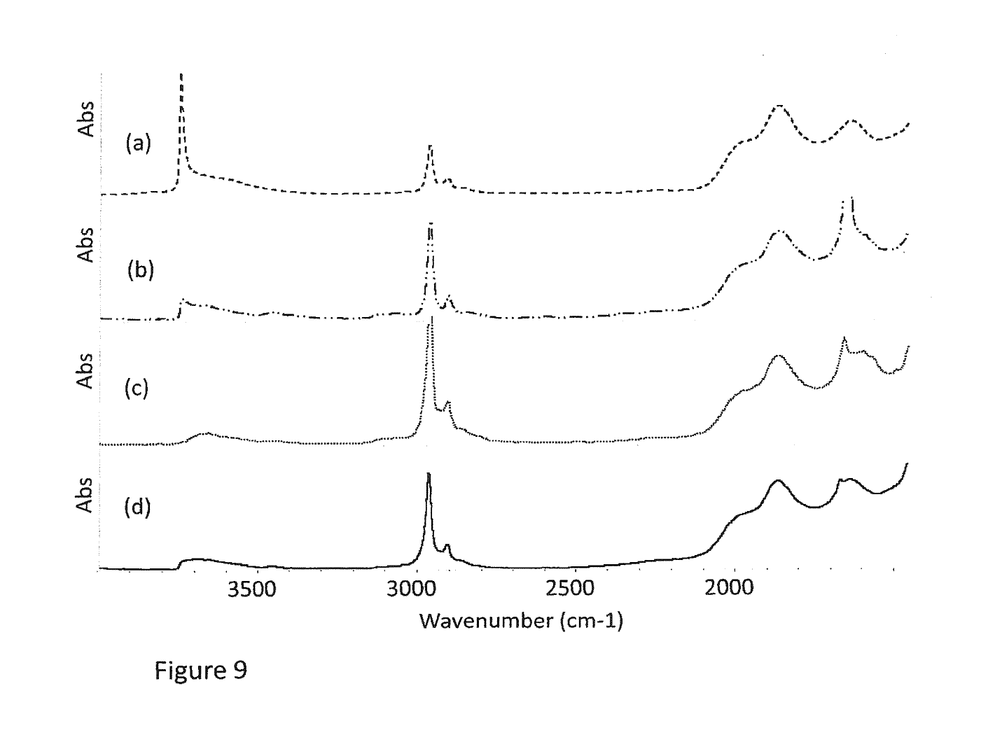

FTIR of the materials [(≡SiO)₂WO₂](Red1)₁, (a), [(≡SiO)₂WO₂](Red2)₁, (b), [(≡SiO)₂WO₂](Red3)₁, (c), and [(≡SiO)₂WO₂](Red4)₁, (d) are shown in FIG. 9.

Reduction of [(≡SiO)₂WO₂] with Different Equiv. of Reductant Red4:

The reductions were carried out following the procedure above. 100 mg of [(≡SiO)₂WO₂] were reduced with various amounts of reductant Red4. Analyses of the filtrate by NMR are summarized in Table 7:

TABLE 7

| Equiv. of | Consump- | Aroma- | | |
|---|---|---|---|---|
| Red4 per | tion | tized | | |
| W center | of Red4 | Bp (Ar) | HMDSO | Material name |
| 0.5 | 100% | 29% | 3% | [(≡SiO)₂WO₂](Red4)_{0.5} |
| 0.8 | 100% | 40% | 3% | [(≡SiO)₂WO₂](Red4)_{0.8} |
| 0.9 | 100% | 46% | 3% | [(≡SiO)₂WO₂](Red4)_{0.9} |
| 1 | 100% | 55% | 3% | [(≡SiO)₂WO₂](Red4)₁ |
| 2 | 68% | 50% | 6% | [(≡SiO)₂WO₂](Red4)₂ |
| 3 | 62% | 29% | 4% | [(≡SiO)₂WO₂](Red4)₃ |
| 4 | 60% | 26% | 3% | [(≡SiO)₂WO₂](Red4)₄ |

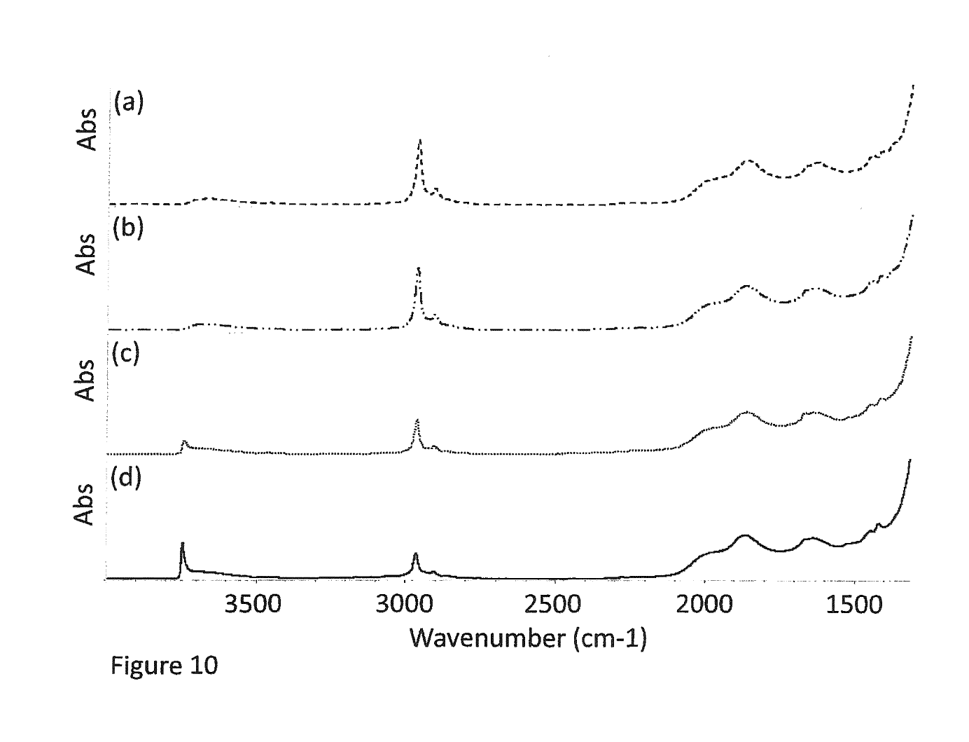

FTIR of the materials [(≡SiO)₂WO₂](Red4)_{0.5}, (d), [(≡SiO)₂WO₂](Red4)₁, (c), [(≡SiO)₂WO₂](Red4)₂, (b), and [(≡SiO)₂WO₂](Red4)₃, (a) are shown in FIG. 10.

B) II) Synthesis of the Molecular Precursors without Involving Alkoholate Comprising Precursors:
Synthesis of WO₂Cl₂(DME)/SiO₂

A solution of 117.6 mg of WO₂Cl₂(DME) (0.312 mmol, 1.2 equiv.) in benzene (4 mL) was added to a suspension of SiO₂₋₍₇₀₀₎ (1 g, 0.26 mmol, 1 equiv.) in benzene (3 mL) at room temperature. The suspension was slowly stirred at room temperature for 12 h. The light green solid was collected by filtration, and was washed by five suspension/filtration cycles in benzene (5×3 mL). The resulting solid was dried thoroughly under high vacuum (10⁻⁵ mbar) at room temperature for 3h to afford 1.05 g of the title compound. All the filtrate solutions were collected and analyzed by ¹H NMR spectroscopy in C₆D₆ using ferrocene as internal standard, indicating that 0.072 mmol of WO₂Cl₂(DME) and 0.096 mmol of DME were released upon grafting.

Elemental Analysis: W 4.35%, C 0.75%, H 0.4% corresponding to 3 C/W (4 expected for the DME adduct), 12 H/W (10 expected for the DME complex).

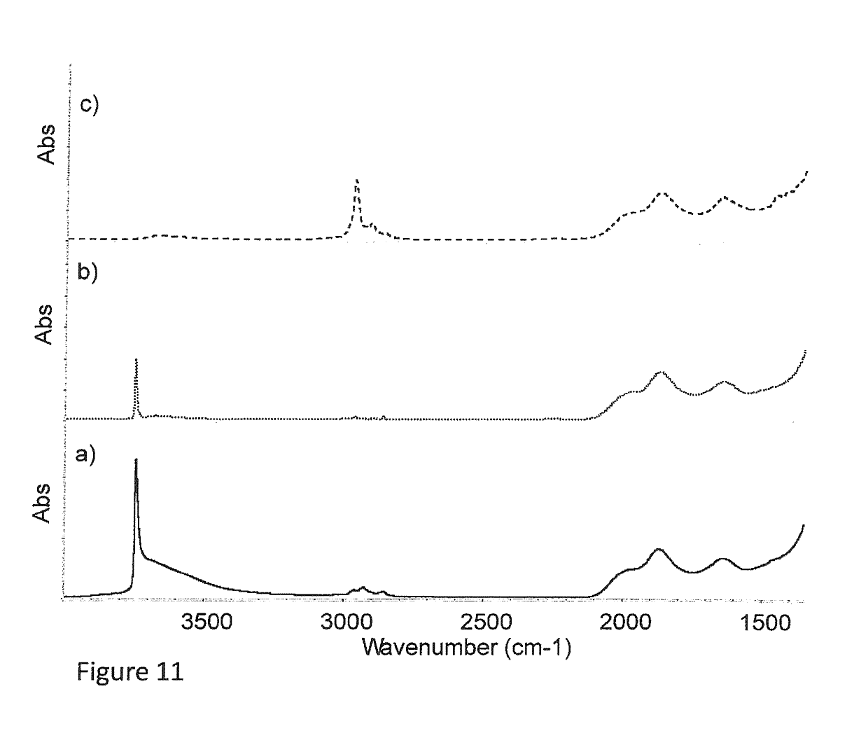

The FTIR transmission spectra of WO₂Cl₂(DME)/SiO₂ is shown in FIG. 11(a).

Thermal Decomposition of WO₂Cl₂(DME)/SiO₂:

Preparation of [(SiO)₂WO₂]_{Cl} (in this and following formulas the index _{Cl} designates that the catalyst has been obtained using a chloride comprising precursor)

WO₂Cl₂(DME)/SiO₂ (1.0 g) was loaded into a reactor and placed under high vacuum (10⁻⁵ mbar) and heated to 200° C. (1° C./min) and kept at 200° C. for 3 h, then heated to 400° C. (1° C./min) and kept at 400° C. for 12 h The reactor was cooled to ambient temperature under vacuum, and [(≡SiO)₂WO₂]_{Cl} was stored in an Ar filled glovebox.

Elemental analysis: W 4.56%.

The FTIR transmission spectra of [(≡SiO)₂WO₂]_{Cl} is shown in FIG. 11(b).

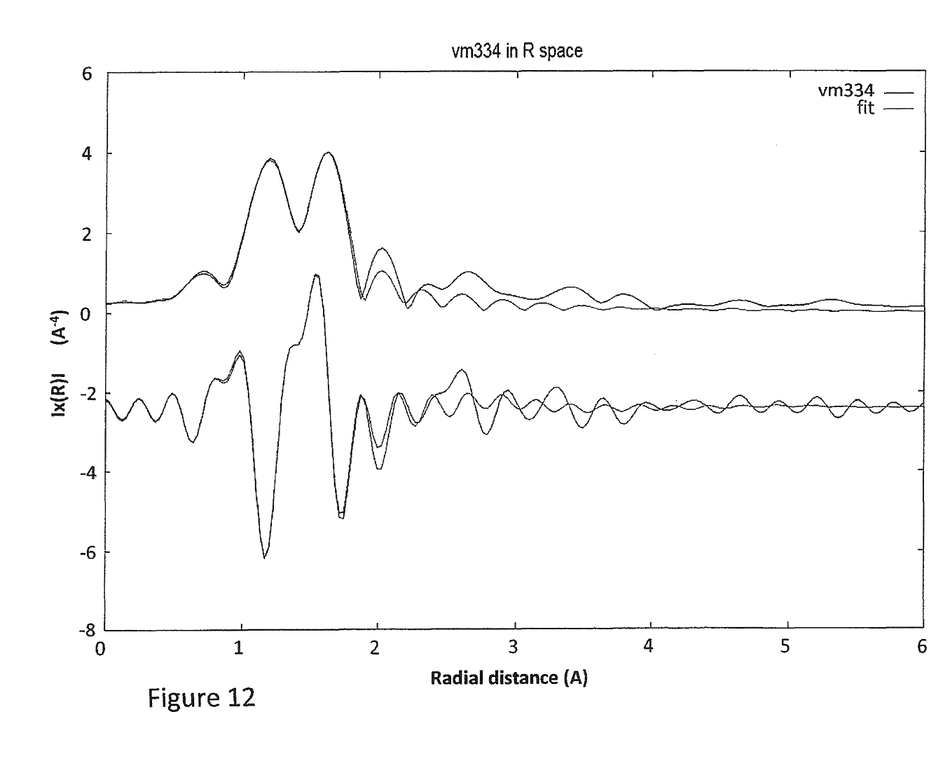

An EXAFS (extended X-ray absorption fine structure) spectrum of WO₂Cl₂(DME) grafted and thermally decomposed onto [SiO₂₋₇₀₀], [(≡SiO)₂WO₂]_{Cl}, is shown in FIG. 12 and the relevant data are listed below in Table 8.

TABLE 8

| Scatterer | N | S02 | r model | delr | R | ss\hat{ }2 | enot |
|---|---|---|---|---|---|---|---|
| O | 2 | 0.852 | 1.6879 | 0.002826 | 1.716 | 0.00311 | 4.524 |
| O | 2 | 0.852 | 1.900 | −0.00480 | 1.895 | 0.00131 | 4.524 |

Reduction of [(≡SiO)₂WO₂]_{Cl} with 1 Equiv. of Reductant Red4:

The reductions were carried out following the procedure described for [(≡SiO)₂WO₂](Red4)₂. 100 mg of [(≡SiO)₂WO₂]_{Cl} were reduced with 2 equiv. of the reductant Red4. Analysis of the filtrate by NMR is given in table 9: The FTIR transmission spectra of [(≡SiO)₂WO₂]_{Cl}(Red4)₂ is shown in FIG. 11(c).

TABLE 9

| | | | | Colour | |
|---|---|---|---|---|---|
| | Consumption | Aromatized | | of the | |
| Reductant | of Red. | Bp (Ar) | HMDSO | material | Material name |
| Red4 | 64% | 48% | 7% | Dark | [(≡SiO)₂WO₂]_{Cl}(Red4)₂ |
| | | | | violet | |

### Preparation of [(≡SiO)MoO₂]:

Grafting of MoO₂[OSi(O^{t}Bu)₃]₂ on SiO₂₋₇₀₀ with DME

A solution of MoO₂[OSi(O^{t}Bu)₃]₂ (301 mg, 0.46 mmol) and DME (0.3 mL) in benzene (10 mL) was added slowly to a suspension of SiO₂₋₇₀₀ (1.71 g, 0.44 mmol SiOH) in benzene (5 mL). The mixture was stirred for 1 day at room temperature and then turned light yellow. The solution was decanted and the solid was washed with benzene four times. All the filtrate solutions were collected and analyzed by ¹H NMR spectroscopy in C6D6 using ferrocene as internal standard, indicating that 0.28 mmol of MoO₂[OSi(O^{t}Bu)₃]₂ and 0.14 mmol of HOSi(OtBu)₃ were present in the filtrate after grafting. Drying the solid obtained under high vacuum for 5 h afforded [(≡SiO)MoO₂{OSi(O^{t}Bu)₃}] as a white solid (1.83 g).

Elemental Analysis: Mo 1.03%, C 1.25%, H 0.29% corresponding to 10 C/W (12 expected), 27 H/W (27 expected).

Thermal Decomposition of [(≡SiO)MoO₂{OSi(O⁵Bu)₃}]

[(≡SiO)MoO₂{OSi(O⁵Bu)₃}] (1.0 g) was loaded into a reactor and placed under high vacuum (10⁻⁵ mbar) and heated to 200° C. (1° C./min) and kept at 200° C. for 3 h, then heated to 400° C. (1° C./min) and kept at 400° C. for 12 h. The color of the solid changed to light gray. The solid was thermally treated in dry air (0.3 atm) at 300° C. for 3 h to afford [(≡SiO)MoO₂] as a white solid. The reactor was cooled to ambient temperature under vacuum, and [(≡SiO)MoO₂] was stored in an Ar filled glovebox.

Elemental Analysis: Mo 1.22%

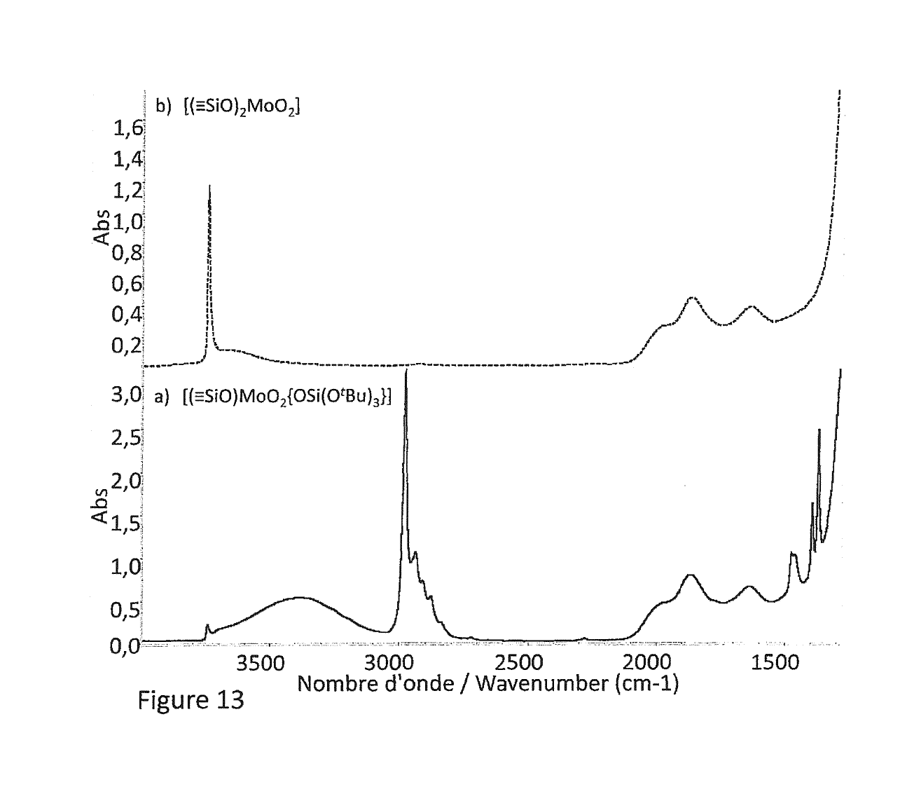

The FTIR transmission spectra of [(≡SiO)MoO₂{OSi(O^{t}Bu)₃}] and [(≡SiO)MoO₂] are shown in FIGS. 13(a) and 13(b), respectively.

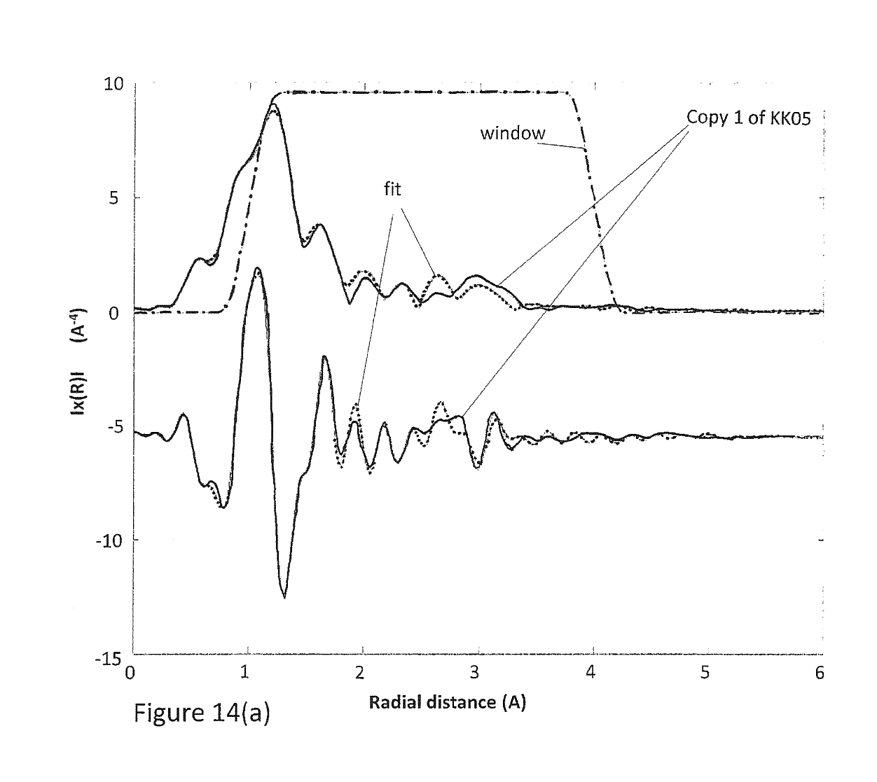

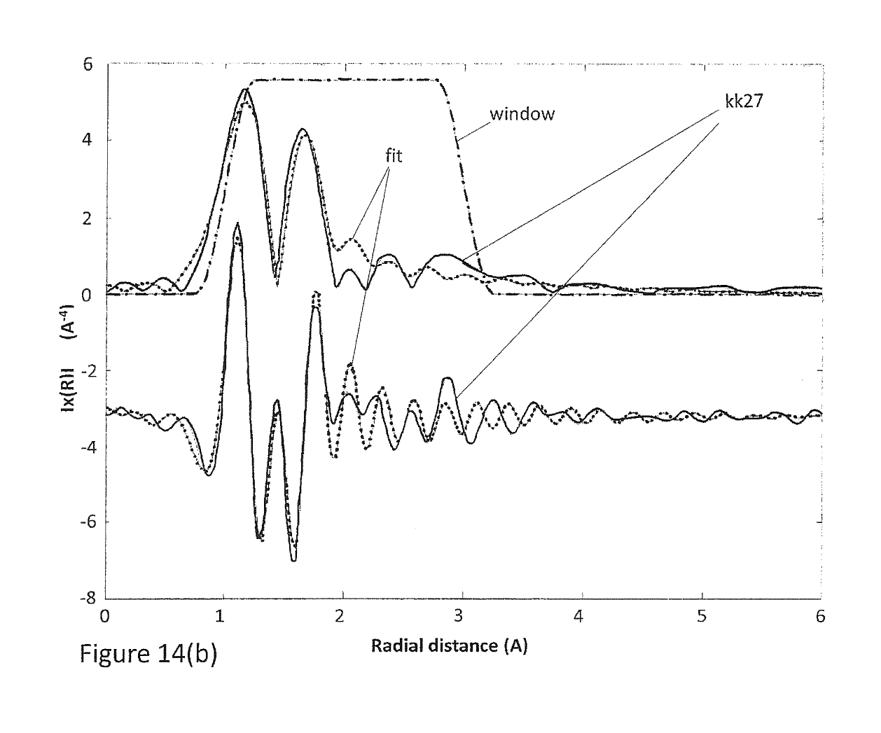

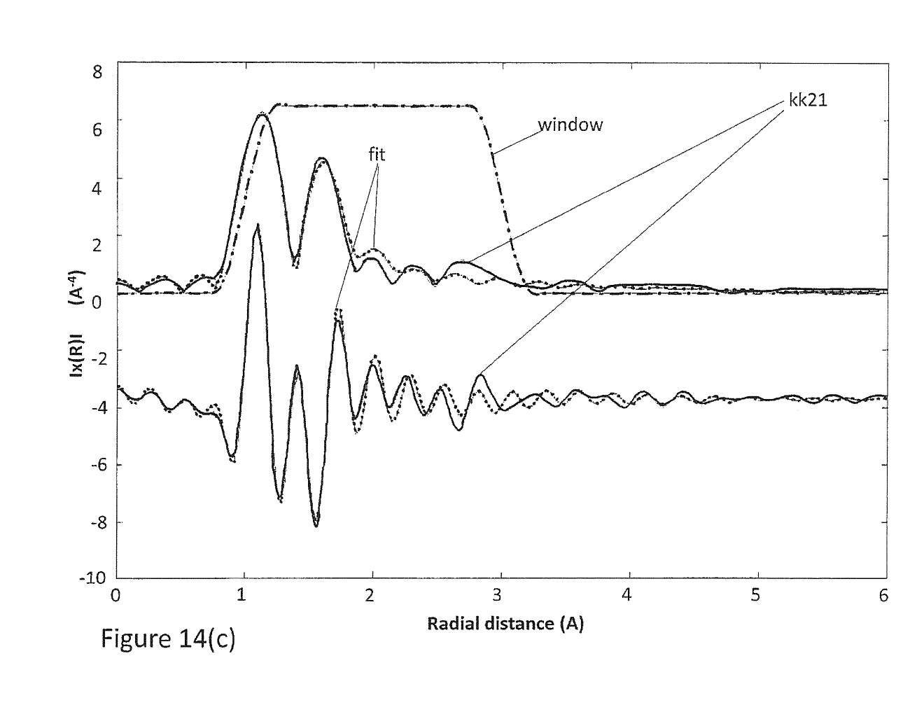

EXAFS (extended X-ray absorption fine structure) spectra of MoO₂[OSi(O^{t}Bu)₃]₂, [(≡SiO)MoO₂{OSi(O^{t}Bu)₃}] and [(≡SiO)MoO₂] are shown in FIG. 14 and the relevant data are listed below in Table 10.

TABLE 10

| | | | | | | | |
|---|---|---|---|---|---|---|---|
| Scatterer | N | S02 | r model | delr | R | ss\hat{ }2 | enot |

MoO₂[OSi(O^{t}Bu)₃]₂

| | | | | | | | |
|---|---|---|---|---|---|---|---|
| O1.1 | 2 | 1.145 | 1.6904 | 0.0066 | 1.697 | 0.00087 | 6.439 |
| O2.1 | 2 | 1.145 | 1.8159 | 0.04519 | 1.86109 | 0.00087 | 6.439 |
| Si1.1 | 2 | 1.145 | 3.4483 | 0.16669 | 3.61499 | 0.01244 | 6.439 |
| O2.1 Si1.1 | 4 | 1.145 | 3.4659 | −0.01243 | 3.45347 | 0.02524 | 6.439 |
| O2.1 Si1.1 | | | | | | | |
| O2.1 | 2 | 1.145 | 3.4836 | −0.01243 | 3.47117 | 0.02524 | 6.439 |

[(≡SiO)MoO₂{OSi(O^{t}Bu)₃}]

| | | | | | | | |
|---|---|---|---|---|---|---|---|
| O | 2 | 1.145 | 1.6904 | 0.04948 | 1.739 | 0.00232 | 4.616 |
| O | 2 | 1.145 | 1.8159 | 0.14283 | 1.958 | 0.00232 | 4.616 |

[(≡SiO)₂MoO₂]

| | | | | | | | |
|---|---|---|---|---|---|---|---|
| O | 2 | 1.145 | 1.6904 | 0.01842 | 1.708 | 0.00074 | −1.995 |
| O | 2 | 1.145 | 1.8159 | 0.1007 | 1.917 | 0.00074 | −1.995 |

Reduction of [(≡SiO)₂MoO₂] with 2 equiv. of Reductant Red4:

The reductions were carried out following the procedure described for [(≡SiO)₂WO₂](Red4)₂. 265 mg of [(≡SiO)₂MoO₂] were reduced with 2 equiv. of the reductant Red4. Analysis of the filtrate by NMR is given in Table 11.

TABLE 11

| | | | | Colour | |
|---|---|---|---|---|---|
| | Consumption | Aromatized | | of the | |
| Reductant | of Red. | Bp (Ar) | HMDSO | material | Material name |
| Red4 | 99% | 70% | 6.5% | Dark | [(≡SiO)₂MoO₂](Red4)₂ |
| | | | | violet | |

Preparation of Re₂O₇/SiO₂

As already indicated in the general procedures, the rhenium/silica was prepared according to the literature procedure described in [7]

### C) Catalytic Activity

### EXAMPLE 1

At t=0 a solution of cis-non-4-ene in toluene was introduced in a glass vial containing [(m5i0)₂WO₂](Red4)₂ produced as described above with a molar ratio of alkene:metal centers of 1000:1. The reaction mixture was stirred at 70° C.; 5 μL aliquots of the solution were sampled and the reaction products over time were analysed. Full conversion was observed in less than 12h, with >99% selectivity.

### EXAMPLE 2

At t=0 a 0.97 M solution of cis-non-4-ene in toluene (339 μL) containing heptane as internal standard (0.11 M) and 2 equivalents of Red4 (with respect to W centers, 0.658 μmol, 0.185 mg) was added to 1.7 mg (0.329 μmol) of the catalyst [(≡SiO)₂WO₂] introduced in a conical base vial containing a wing shaped magnetic stirring bar. The reaction mixture was stirred at 600 rpm and kept at 70° C. using an aluminum heating block. 5 μL aliquots of the solution were sampled, diluted with pure toluene (100 μL) and quenched by the addition of 1 μL of wet ethyl acetate. The resulting solution was analyzed by GC/FID (Agilent Technologies 7890 A) equipped with an HP-5 (Agilent Technologies) column.

Full conversion was observed in less than 3h, with >99% selectivity.

### EXAMPLE 3

In a manner similar to the one described in Example 1, cis-4-nonene (1000 equivalents) was metathesized using [(≡SiO)₂WO₂](Red1)_{0.5} instead of [(≡SiO)₂WO₂](Red4)₂.

Full conversion was observed in less than 12h, with >99% selectivity.

### EXAMPLE 4

In a manner similar to the one described in Example 2 and using 100 equivalents (with respect to the tungsten centres) of ethyl oleate in toluene and 2 equivalents of Red4 with 1 equivalent of [(≡SiO)₂WO₂], ethyl oleate (100 equivalents) was metathesized to full conversion in less than 24h, with >99% selectivity.

### EXAMPLE 5

### Examples 2 and 4 were repeated except that no organosilicon reductant was added to the reaction mixture. No reaction products could be observed after 24h.

The above data clearly demonstrate the significant advantage obtained with the catalysts treated with the organosilicon reductants; an unactivated tungsten oxide catalyst did not show any activity in the conditions tested above, while catalysts treated with organosilion reductants demonstrated high activity in alkene metathesis. Highest activity was obtained when the organosilicon reagent was added together with the olefin substrate although independant reduction also resulted in increased activity.

### EXAMPLE 6

### Investigation of Different Precursors

### a) Molecular Precursors

In a manner similar to the one described in Example 2, cis-4-nonene (1000 equivalents) was metathesized using toluene solutions of one equivalent of molecular precursors (given below) treated with two equivalents of Red4 at 70° C. Conversions are reported in Table 12. For all the precursors listed in Table 8, no activity was observed in absence of reductant.

TABLE 12

| | TOF_{3 min} | TOFₘₐₓ | Conversion |
|---|---|---|---|
| Catalyst | (min⁻¹) | (min⁻¹)^{a} | at 24 h |
| WCl₆ | <0.1 | <0.1 | 2.6% ^{b} |
| WOCl₄ | <0.1 | <0.1 | >1% ^{b} |
| WO₂Cl₂(DME) | <0.1 | <0.1 | 1.5% |
| WO(OSi(OtBu)₃)₄ | <0.1 | <0.1 | >1% |
| WO₂(OSi(OtBu)₃)₂(DME) | <0.1 | <0.1 | >1% |

^{a}Maximum TOF (turn over frequency) determined during the test. Values in bracket are the time for which maximum TOF was observed.
^{b} Full isomerisation of the substrate to thermodynamic Z/E ratio was observed with this substrate.

### b) Heterogeneous Catalysts

In a manner similar to the one described in example 2, cis-4-nonene (1000 equivalents with respect to metal centers) was metathesized using heterogeneous catalysts (given below) treated with two equivalents of Red4 (per metal centers). Conversions are reported in Table 13. For all the precursors listed in Table 13, negligible activity was observed in the absence of reductant.

TABLE 13

| | TOF_{3 min} | TOFₘₐₓ | Time to | |
|---|---|---|---|---|
| Catalyst (0.1 mol % W) | (min⁻¹) | (min⁻¹)^{a} | conversion | |
| [(≡SiO)₂WO₂] | 3 | 8 (10 min) | 3 | h |
| WO₃/SiO₂ | 1 | 2 (320 min) | 24 | h |
| MoO₃/SiO₂ | 0.5 | 0.6 (60 min) | 24 | h |

| | | | |
|---|---|---|---|
| [(≡SiO)WO₂(OSi(OtBu)₃)] | 0.3 | 0.3 (3 min) | 10% |
| | | | conversion |
| | | | after 24 h |

^{a}Maximum TOF (turn over frequency) determined during the test. Values in bracket are the time for which maximum TOF was observed.

### EXAMPLE 7

### Metathesis of cis-non-4-ene by Pre-Reduced Materials [(≡SiO)₂WO₂](Redn)ₓ

At t=0 a 0.97 M solution of cis-non-4-ene in toluene (379 μL for [(≡SiO)₂WO₂](Red 1)₁, 539 μL for [(≡SiO)₂WO₂](Red 4)_{0.5}, 339 μL for [(≡SiO)₂WO₂](Red 4)₁, 399 μL for [(≡SiO)₂WO₂](Red 4)₂) containing heptane as internal standard (0.11 M) was added to the catalyst ((1.9 mg of [(≡SiO)₂WO₂](Red 1)₁, 2.7 mg of [(≡SiO)₂WO₂](Red 4)_{0.5}, 1.7 mg of [(≡SiO)₂WO₂](Red 4)₁ or 2.0 mg of [(≡SiO)₂WO₂](Red 4)₂) introduced in a conical base vial containing a wing shaped magnetic stirring bar. The reaction mixture was stirred at 600 rpm and kept at 70° C. using an aluminum heating block. 5 μL aliquots of the solution were sampled, diluted with pure toluene (100 μL) and quenched by the addition of 1μL of wet ethyl acetate. The resulting solution was analyzed by GC/FID (Agilent Technologies 7890 A) equipped with an HP-5 (Agilent Technologies) column. The results are listed in Table 14.

TABLE 14

| | TOF_{3 min} | TOFₘₐₓ | Time to final | |
|---|---|---|---|---|
| Catalyst (0.1 mol % W) | (min⁻¹) | (min⁻¹)^{a} | conversion | |
| [(≡SiO)₂WO₂](Red1)₁ | 17 | 17 (3 min) | 12 | h |
| [(≡SiO)₂WO₂](Red4)_{0.5} | 5 | 8 (10 min) | 3 | h |
| [(≡SiO)₂WO₂](Red4)₁ | 2 | 3 (10 min) | 6 | h |
| [(≡SiO)₂WO₂](Red4)₂ | <1 | 2 (540 min) | 12 | h |

^{a}Maximum TOF determined during the test. Values in bracket give the time at which maximum TOF was observed.

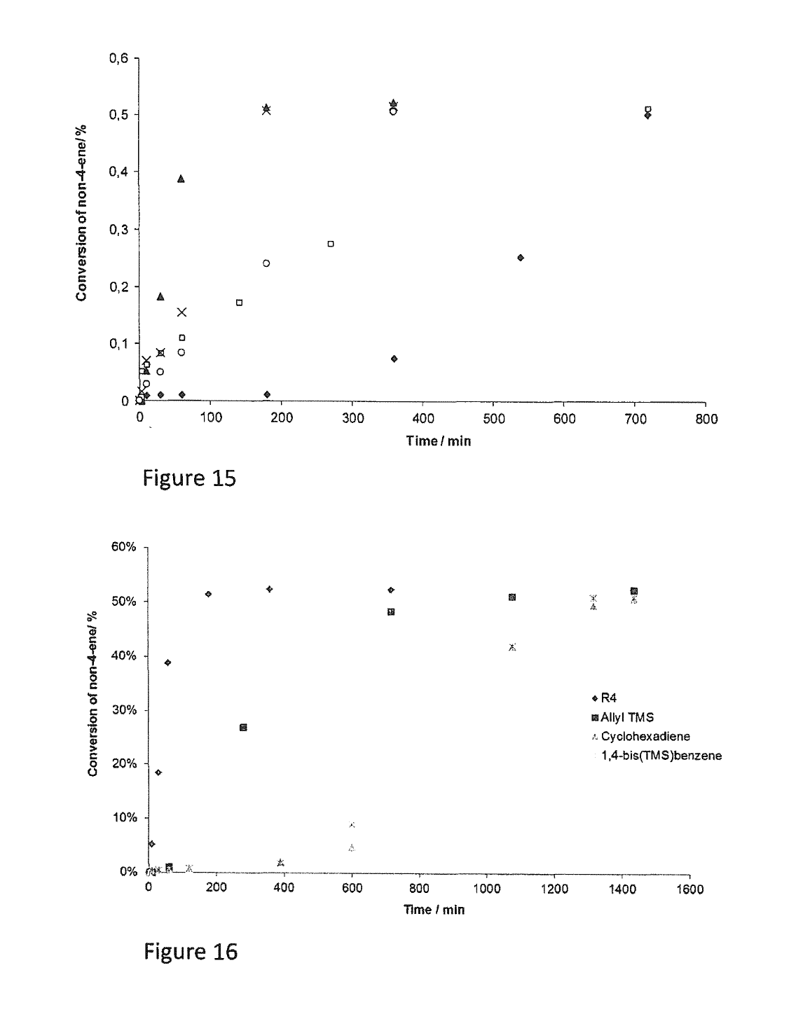

A visual presentation of conversion vs time of cis-4-nonene homometathesis using 0.1 mol % W, 70° C. is given in FIG. 15 for [(≡SiO)₂WO₂] (Red4)₂ (diamonds), [(≡SiO)₂WO₂] (Red4)₁ (empty circles), [(≡SiO)₂WO₂] (Red4)_{0.5} (crosses), [(≡SiO)₂WO₂](Red1)₁ (empty squares) and [(≡SiO)₂WO₂]+0.2 mol % Red4 (triangles)

### EXAMPLE 8

### Metathesis of Functionalized Olefins by [(≡SiO)₂WO₂] in Presence of 2 Equiv. of Red4 at 70° C.

Following the procedure described in Examples 2 and 4, metathesis of further olefin substrates has been investigated. The results are listed in Table 15.

TABLE 15

| | | TOFₘₐₓ | Time to final | |
|---|---|---|---|---|
| Substrate | Mol % | (min⁻¹) | conversion | |
| Cis-4-nonene | 0.1 | 8 (10 min) | 3 | h |
| Ethyl Oleate | 1 | 4 (3 min) | <24 | h |
| Cyclooctene | 1 | 10 (5 min) | 20 | min |

| | | | |
|---|---|---|---|
| Diethyl Diallylmalonate | 1 | <0.1 | 15% at 24 h |
| Phenylpropyne | 1 | <0.1 | 7% at 24 h |

### EXAMPLE 9

### Metathesis of cis-non-4-ene by [(≡SiO)₂WO₂] 0.1 mol % in Presence of 2 Equiv. of Other Reagents (toluene, 70° C.).

Metathesis of cis-non-4-ene with organic reductants different from organosilicon reductants of Formula (II) has been performed as described in Example 2, using [(≡SiO)₂WO₂], 0.1 mol % in the presence of 2 equiv. of reductant. The results are shown in Table 16.

TABLE 16

| | TOFₘₐₓ | Time to final | |
|---|---|---|---|
| reagent | (min⁻¹) | conversion | |
| Red4 | 8 (10 min) | 3 | h |
| allylTMS | 4 (3 min) | 24 | h |
| cyclohexadiene | 0.8 (18 h) | 24 | h |
| 1,4-bis(TMS)benzene | 0.6 (18 h) | 22 | h |

A visual presentation of conversion vs time of cis-4-nonene homometathesis, 0.1 mol % W, 70° C. for [(≡SiO)₂WO₂] in presence of two equivalents of the following reagents: Red4 (diamonds), allyltrimethyilane (squares), cyclohexadiene (triangles), and 1,4-bistrimethylsilylbenzene (stars) in FIG. 16.

### EXAMPLE 10

### Recycling of Spent [(≡SiO)₂WO₂] Catalyst with 2 Equiv. of Red4 at 70° C.

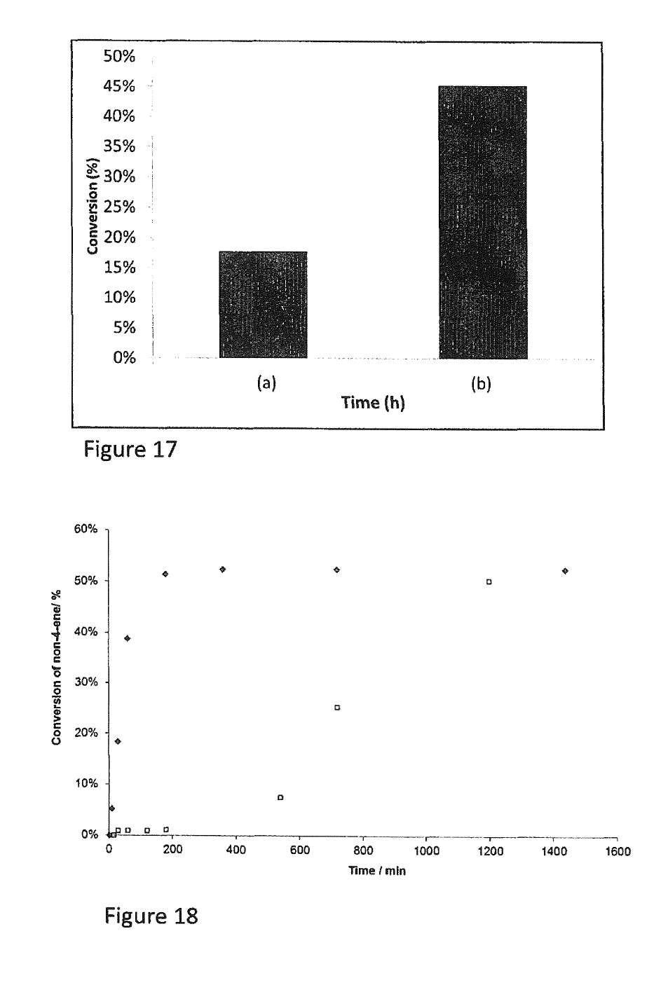

Metathesis of diethyl diallylmalonate was carried out following the procedure described in Example 8 (with 1. mol % catalyst [((≡SiO)₂WO₂], 2 equiv. of Red4, 70° C., in toluene). After 90 h, 18% conversion was observed but no activity could be further detected. To this deactivated catalyst were added two equivalents of Red4, reinitiating catalytic activity, to reach 45% conversion 90h after the addition. The results are presented in FIG. 17: conversion 90h after initial Red4 addition (a) and 90h after second addition of 2 equiv. of Red4 (b).

### EXAMPLE 11

At t=0 a 0.81 M solution of cis-non-4-ene in toluene (457 μL) containing heptane as internal standard (0.10 M) and 2 equivalents of Red4 (with respect to W centers, 0.744 μmol, 0.210 mg) was added to 1.5 mg (0.372 μmol) of the catalyst [((≡SiO)₂WO₂]_{Cl} introduced in a conical base vial containing a wing shaped magnetic stirring bar. The reaction mixture was stirred at 600 rpm and kept at 70° C. using an aluminum heating block. 5 μL. aliquots of the solution were sampled, diluted with pure toluene (100 μL) and quenched by the addition of 1μL of wet ethyl acetate. The resulting solution was analyzed by GC/FID (Agilent Technologies 7890 A) equipped with an HP-5 (Agilent Technologies) column.

Conversion to the thermodynamic equilibrium was observed in less than 3h, with >99% selectivity. A plot of conversion vs. time is given in FIG. 18.

### EXAMPLE 12

At t=0 a 0181 M solution of cis-non-4-ene in toluene (488 μL) containing heptane as internal standard (0.10 M) was added to 1.6 mg (0.396 μmol) of the catalyst [((≡SiO)₂WO₂]_{Cl}(Red4)₂ introduced in a conical base vial containing a wing shaped magnetic stirring bar. The reaction mixture was stirred at 600 rpm and kept at 70° C. using an aluminum heating block. 5 μL aliquots of the solution were sampled, diluted with pure toluene (100 μL) and quenched by the addition of 1 μL of wet ethyl acetate. The resulting solution was analyzed by GC/FID (Agilent Technologies 7890 A) equipped with an HP-5 (Agilent Technologies) column.

Full conversion was observed in less than 24h, with >99% selectivity. A plot of conversion vs. time is given in FIG. 18.

### EXAMPLE 13

### Ethyl oleate Self-Metathesis with [((≡SiO)₂WO₂]_{Cl}

In a manner similar to the one described in Example 2 and using 100 equivalents (with respect to the tungsten centres) of ethyl oleate in toluene and 2 equivalents of Red4 with 1 equivalent of [((≡SiO)₂WO₂Cl], ethyl oleate (100 equivalents) was converted to the thermodynamic equilibrium in less than 24h, with >99% selectivity.

### EXAMPLE 14

### Butene/ethylene Cross-Metathesis with [(≡SiO)₂WO₂](Red4)₂

A pellet of the solid [(SiO)₂WO₂](Red4)₂ (5.4 μmol) was loaded in a flow reactor in the glove box, the isolated reaction chamber was then connected to the gas line. Tubes were flushed with the gas mixture (butene:ethylene:nitrogen 1:1:12 mol ratio) for 2 h. Before opening to the reaction chamber, the flow rate was set to 60 μmol/min for both ethylene and butene (11 mol alkene.mol_{w}⁻¹.min⁻¹), the temperature was set to 100° C. The opening of the valve corresponds to the beginning of the catalysis and the reaction was monitored by GC using an auto-sampler. 13% conversion was observed after 3h reaction time with 99% selectivity for propene formation.

### EXAMPLE 15

### Ethyl oleate ethenolysis

A 1 mL of ethyl oleate containing octadecane as internal standard was added to 72 mg (14 μmol) of the catalyst [((≡SiO)2WO₂] in a 10 mL vial and pressurized with 10 Bar ethylene. The reaction mixture was stirred at 600 rpm and kept at 80° C. during the reaction. At t=0, 2 a solution of 2 equivalents of Red4 in 1 mL toluene was added to the reaction mixture. After 24h reaction, the catalyst was quenched by addition of 100 μL of wet ethyl acetate. The resulting solution was analyzed by GC/FID (Agilent Technologies 7890 A) equipped with an HP-88 (Agilent Technologies) column. The catalyst reached 20% conversion with 92% selectivity for the ethenolysis products.

### EXAMPLE 16

At t=0 a 0.95 M solution of cis-non-4-ene in toluene (400 μL) containing heptane as internal standard (0.10 M) and 2 equivalents of Red4 (with respect to Mo centers, 0.762 μmol, 0.220 mg) was added to 3 mg (0.380 μmol) of the catalyst [((≡SiO)₂MoO₂] introduced in a conical base vial containing a wing shaped magnetic stirring bar. The reaction mixture was stirred at 600 rpm and kept at 30° C. using an aluminum heating block. 5 μL aliquots of the solution were sampled, diluted with pure toluene (100 μL) and quenched by the addition of 1 μL of wet ethyl acetate. The resulting solution was analyzed by GC/FID (Agilent Technologies 7890 A) equipped with an HP-5 (Agilent Technologies) column.

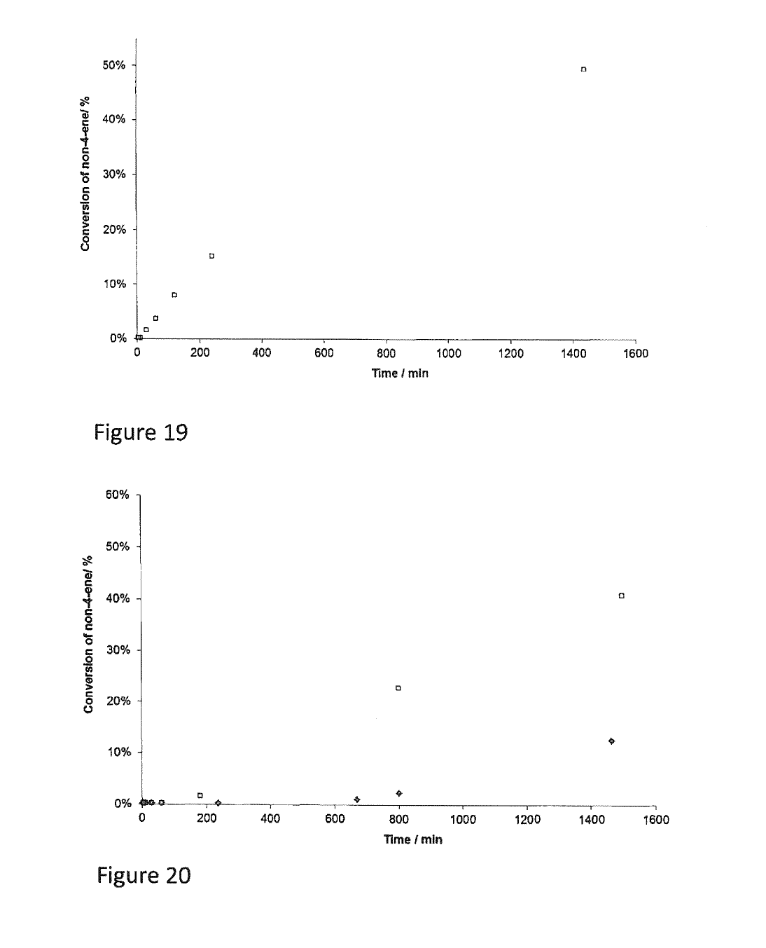

Full conversion was observed in less than 24h, with >99% selectivity. A plot of conversion vs. time is given in FIG. 19. When a similar test is carried out in the absence of reductant, no catalytic activity is observed.

### EXAMPLE 17

In Absence of Reductant:

At t=0 a 0.95 M solution of cis-non-4-ene in toluene (401 μL) containing heptane as internal standard (0.10 M) was added to 1.4 mg (0.39 μmol) of the catalyst Re₂O₇/SiO₂ introduced in a conical base vial containing a wing shaped magnetic stirring bar. The reaction mixture was stirred at 600 rpm and kept at 70° C. using an aluminum heating block. 5 μL aliquots of the solution were sampled, diluted with pure toluene (100 μL) and quenched by the addition of 1 μL of wet ethyl acetate. The resulting solution was analyzed by GC/FID (Agilent Technologies 7890 A) equipped with an HP-5 (Agilent Technologies) column. 12% conversion was observed in 24h, with >90% selectivity. A plot of conversion vs. time is given in FIG. 20.

In Presence of Two Equivalents of Red1:

At t=0 a 0.95 M solution of cis-non-4-ene in toluene (573 4) containing heptane as internal standard (0.10 M) and 2 equivalents of Red1 (with respect to Re centers, 1.1 μmol, 0.26 mg) was added to 2.0 mg (0.55 μmol) of the catalyst Re₂O₇/SiO₂ introduced in a conical base vial containing a wing shaped magnetic stirring bar. The reaction mixture was stirred at 600 rpm and kept at 70° C. using an aluminum heating block. 5 μL aliquots of the solution were sampled, diluted with pure toluene (100 μL) and quenched by the addition of 1 μL of wet ethyl acetate. The resulting solution was analyzed by GC/FID (Agilent Technologies 7890 A) equipped with an HP-5 (Agilent Technologies) column. 41% conversion was observed in 24h, with >90% selectivity. A plot of conversion vs. time is given in FIG. 20.

### EXAMPLE 18

Neat 9-methyl decenoate (310 μL., 1.48 mmol) was added to 5.3 mg (2.8 μmol) of the catalyst MoO₃/SiO₂ introduced in a vial containing a magnetic stirring bar. At t=0, 2 equivalents of Red4 (with respect to Mo centers, 5.6 μmol, 1.6 mg, as 0.1 M solution in toluene) was added to the reaction mixture. The reaction mixture was stirred at 100 rpm and kept at 150° C. using an aluminum heating block.

57% conversion was observed in less than 24h, with >99% selectivity. When a similar test is carried out in the absence of reductant, no catalytic activity is observed.

### EXAMPLE 19

Neat 9-methyl dodecenoate (E/Z=85/15) (355 μL, 1.45 mmol) was added to 5.3 mg (2.8 μmol) of the catalyst MoO₃/SiO₂ introduced in a vial containing a magnetic stirring bar. At t=0, 2 equivalents of Red4 (with respect to Mo centers, 5.6 μmol, 1.6 mg, as 0.1 M solution in toluene) was added to the reaction mixture. The reaction mixture was stirred at 100 rpm and kept at 150° C. using an aluminum heating block.

22% conversion was observed in less than 24h, with 94% selectivity. When a similar test is carried out in the absence of reductant, no catalytic activity is observed.

### REFERENCES

[1] Dreisch, K., et al., Polyhedron 1991, 10 (20-21), 2417-2421.
[2] Gibson, V. C., et al., Polyhedron 1990, 9 (18), 2293-2298.
[3] Laguerre, M., et al., J. Organomet. Chem. 1975, 93 (2), C17-C19.
[4] Saito, T., et al., J. Am. Chem. Soc. 2014, 136 (13), 5161-5170.
[5] Ross-Medgaarden, E. I.; Wachs, I. E., The Journal of Physical Chemistry C 2007, 111 (41), 15089-15099.
[6] Jarupatrakorn, J.; et al., Chem. Mater. 2005, 17 (7), 1818-1828.
[7] Duquette, L. G.; Cielinski, R. C.; Jung C. W. and Garrou, P. E., J. Catal. 1984, 90, 362
[8] Schattenmann, W. C., Dissertation, Anorganisches Institut der Technischen Universität München 1997
[9] Marciniec, B.; Foltynovicz, Z.; Lewandowski, M., Journal of Molecular Catalysis 1994, 90, 125-133

1. A method for producing an activated supported catalyst, said method comprising contacting a supported catalyst of the type MOₙEₘ with E being S and/or Se, in particular MO_{D}, with at least one organic reductant, said reductant comprising at least one double bond or at least one silyl group of the type SiX₂Y in such proximity to one or more further double bond(s) that upon oxidation an aromatic structure is formed , wherein in the silyl group of the type SiX₂Y
each X is independently selected from H, R′, halogen, OR, NR₂, wherein
each R′ is independently selected from
unsubstituted or substituted, linear or branched or cyclic C1 to C18 alkyl,
unsubstituted or substituted linear or branched or cyclic C1 to C18 alkenyl,
unsubstituted or substituted linear or branched or cyclic C1 to C18 alkynyl, or
an unsubstituted or substituted aromatic group
each R is independently selected from H, R′, silyl of type —SiX₂Y
the Y of each silyl group can be the same or different and is selected from the group as defined for X or two Y together are —O— or a single bond
in an oxygen-free and dry environment.

2. The method of claim 1, wherein the reductant is a reductant of formula (I) 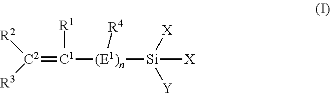
wherein
E¹ is selected from C—R⁵, N, P, As, or B
n is 0 or 1
R¹ to R⁴ and R⁵ are the same or different and are selected from the group comprising —H, —R′, silyl of type —SiX₂Y, —OR, —NR₂, halogens, —NO₂, phosphates, carbonates and sulfates, wherein in all the groups
each R′ is independently selected from the group comprising
unsubstituted or substituted, linear or branched or cyclic C1 to C18 alkyl,
unsubstituted or substituted linear or branched or cyclic C1 to C18 alkenyl,
unsubstituted or substituted linear or branched or cyclic C1 to C18 alkynyl or
an unsubstituted or substituted aromatic group, in particular optionally aryl substituted C1 to C6 alkyl, such as methyl or butyl or benzyl or methylbenzyl, optionally alkyl like methyl substituted cyclohexyl, optionally alkyl like methyl substituted phenyl, e.g. tolyl,
each R is independently selected from the group comprising H, R′, silyl of type SiX₂Y,
or
R¹ and R² together form a —(E²)ₗ— chain that together with the C¹ and C² to which they are bound form a 4- to 12-membered ring, wherein
l is 2 to 10
and/or
R³ and R⁴ together form a —(E²)ₘ— chain that together with the C² and E¹ to which they are bound form a 4- to 12-membered ring, wherein
m is 1 to 9 and wherein
each E² is independently selected from the group comprising E¹R⁶, or O, or two adjacent E² are —CR⁷═CR⁸—, preferably in vinylic or allylic position with regard to one or more SiX₂Y group(s), wherein
E¹ is as defined above
R⁶, R⁷ and R⁸ are as defined for R⁵ or SiX₂Y
each X is independently selected from the group comprising H, R′, halogen, OR, NR₂, wherein
R′ and R are as defined above
each Y is as defined above for X or two Y together are —O— or a single bond, wherein said —X₂Si——SiX₂— groups can be on adjacent E¹ and E² and/or on two adjacent E² and/or on adjacent E¹ and C1 and/or on adjacent E² and C², and/or on C¹ and C², and/or on E¹ and E² spaced further apart and/or on E¹ and C² and/or on E² and C¹ spaced further apart and/or on E² and C² spaced further apart and /or on two E2 spaced further apart.

3. The method of claim 2, wherein at least one of the variables in formula (I) and much preferred all variables are selected from the following groups:
E¹ is selected from C—R⁵ and N
n is 1
R¹ to R⁴ and R⁵ are the same or different and are selected from the group comprising —H, —R′, silyl of type —SiX₃, wherein in all the groups
each R′ is independently selected from the group comprising
unsubstituted or substituted, linear or branched or cyclic C1 to C6 alkyl,
unsubstituted or substituted linear or branched or cyclic C1 to C6 alkenyl,
unsubstituted or substituted linear or branched or cyclic C1 to C6 alkynyl or
an unsubstituted or substituted up to 6 membered aromatic group,
each R is independently selected from the group comprising H, R′, silyl of type —SiX₃,
or
R¹ and R² together form a —(E²)ₗ— chain that together with the C¹ and C² to which they are bound form a 6-membered ring, wherein
l is 4
and/or
R³ and R⁴ together form a —(E²)ₘ— chain that together with the C² and E¹ to which they are bound form a 5 to 8-membered ring, wherein
m is 2 to 5 and wherein
each E² is independently selected from the group comprising E¹R⁶, or two adjacent E² are —R⁷═CR⁸—, preferably in vinylic or allylic position with regard to one or more SiX₃ group(s), wherein
E¹ is as defined above
R⁶, R⁷ and R⁸ are as defined for R⁵ or SiX₃
each X is independently selected from the group comprising H and R′, wherein
R′ is as defined above.

4. The method of any one of claims 1 to 3, wherein at least one reductant is selected from compounds of one of formulas (II) to (VII)

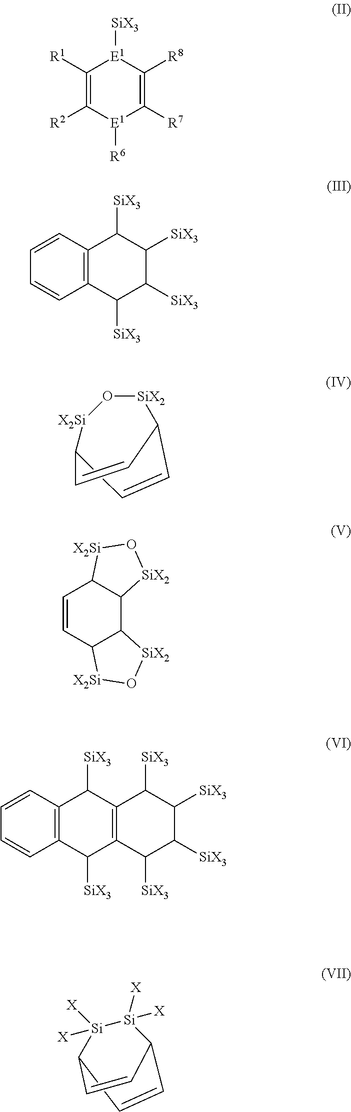

5. The use of any one of claims 1 to 4, wherein at least one reductant is a compound of formula (H), preferably at least one of

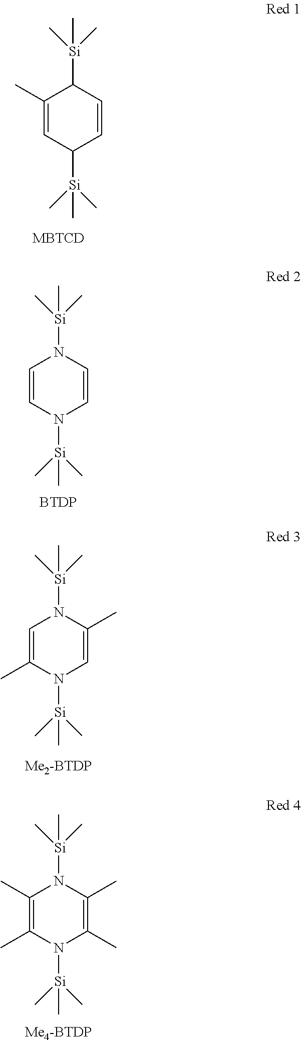

6. The method of any one of claims 1 to 5, wherein the reduction reaction is performed without a solvent or with a solvent, said solvent being an aprotic solvent and/or at a temperature in the range of −20° C. to 500° C., preferably 40° C. to 250° C., more preferred at about 70° C.

7. The method of any one of claims Ito 6, wherein the supported catalyst is of type MOₙEₘ, in particular of the type MOₙ, wherein M is selected from the group consisting of W, Mo, Re and combinations thereof, and wherein the support is a metal oxide, in particular a metal oxide selected from silica, alumina, ceria, titania, zirconia, niobia, thoria or mixed oxides such as Al₂O₃—SiO₂, in particular silica.

8. Use of an organic reductant as defined in any one of claims 1 to 5 for activating a supported catalyst of the type MOₙEₘ, in particular of the type MOₙ, wherein M is selected from the group consisting of W, Mo, Re or combinations, such as a supported WO₃ or MoO₃ or Re₂O₇.

9. The use of claim 8, wherein the support is selected from the group consisting of silica, alumina, ceria, titania, niobia, zirconia, thoria or mixed oxides such as Al₂O₃—SiO₂, in particular silica.

10. A supported catalyst obtainable by the method of any one of claims 1 to 7.

11. A supported catalyst which is an at least partially reduced MOₙ catalyst with the formula (VIII), 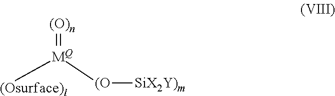
wherein
Q is the valence of the metal which may be a mixed valence due to differently reduced metal centers
l is 1 to 4,
n is 0 to 2,
l+m+2n=Q and
each X is independently selected from H, R′, halogen, OR, NR₂, wherein
each R′ is independently selected from
unsubstituted or substituted, linear or branched or cyclic C1 to C18 alkyl,
unsubstituted or substituted linear or branched or cyclic C1 to C18 alkenyl,
unsubstituted or substituted linear or branched or cyclic C1 to C18 alkynyl, or
an unsubstituted or substituted aromatic group, in particular optionally aryl substituted C1 to C6 alkyl such as methyl or butyl or benzyl or methylbenzyl, optionally alkyl like methyl substituted cyclohexyl, optionally alkyl like methyl substituted phenyl, such as tolyl, and
each R is independently selected from the group consisting of H, R′ and silyl of the type —SiX₂Y, wherein
R′ is as defined above and
each Y can be the same or different and is selected from the group as defined for X or two Y together are —O— or a single bond.

12. Use of a supported catalyst obtained by the method of anyone of claims 1 to 7 or a catalyst of any one of claim 10 or 11 in alkene metathesis, in particular in the metathesis of functionalized alkenes.

13. A method for alkene metathesis comprising contacting a supported MOₙEₘ catalyst, in particular a supported MOₙ catalyst, with a reductant as defined in any one of claims 1 to 5 and contacting said reduced catalyst with an alkene to be metathesized under metathesis conditions.

14. The method of claim 13 wherein the reduction reaction is performed in situ by simultaneously combining supported MOₙEₘ catalyst, in particular supported MOₙ catalyst, reductant and alkene to be metathesized under metathesis conditions.

15. The method of claim 13 or 14, wherein the metathesis conditions are a temperature in the range of −20° C. to 500° C., preferably 40° C. to 250° C., more preferred at about 70° C., and the ratio of alkene substrate to M is 0.90001 to 1 mole of metal per mole of substrate.

16. The method of any one of claims 13 to 15, wherein the catalyst is regenerated in situ by reacting with reductant either separately or in situ according to claim 14. cm 17. A method for producing an activated supported catalyst, said method comprising contacting a supported catalyst of the type MOₙEₘ, in particular of the type MOₙ with at least one organic reductant, said reductant being a compound of formula (I) 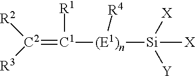
wherein R1 to R4, E1, n, X and Y are as defined in any one of claims 2 to 5, preferably the conditions are as defined in claim 6 and the supported catalyst preferably is as defined in claim 7.
